(19) United States
(12) Patent Application Publication
(10) Pub. No.: US 2025/0387595 A1
(43) Pub. Date: Dec. 25, 2025

(54) INSERT CATHETER WITH PRE-SHAPED TIP

(71) Applicant: Imperative Care, Inc., Campbell, CA (US)

(72) Inventors: Brandon Yee, Oakland, CA (US); Shashi Desai, San Jose, CA (US); Clark Elvin Ibarra Reyes, San Jose, CA (US); Ryan Jeffrey Connolly, San Carlos, CA (US)

(21) Appl. No.: 19/241,196

(22) Filed: Jun. 17, 2025

Related U.S. Application Data
(60) Provisional application No. 63/662,989, filed on Jun. 21, 2024.
(60) Provisional application No. 63/664,675, filed on Jun. 26, 2024.

(51) Int. Cl. A61M 25/00

(52) U.S. Cl. CPC A61M 25/0068; A61M 25/0053; A61M 2210/127

(57) ABSTRACT

A neurovascular catheter may include an elongate flexible body having a length of at least about 130 cm. The catheter can include a distal portion including a pre-shaped tip and having a length between about 2 cm to 9 cm, a transitional portion proximal to the distal portion that is less flexible than the distal portion, wherein the transitional portion has a length of between about 14 cm and about 16 cm; and a proximal portion proximal to the transitional portion that is less flexible than the transitional portion. The proximal portion has a length between about 110 cm and about 115 cm. A flexibility profile of the elongate flexible body is measurable with a cantilever beam test to determine a stiffness measures in peak load value/distance.

### INCORPORATION BY REFERENCE TO ANY PRIORITY APPLICATIONS

Any and all applications for which a foreign or domestic priority claim is identified in the application data sheet as filed with the present application are hereby incorporated by reference under 37 C.F.R. § 1.57. The present application claims priority to U.S. Provisional Patent Application No. 63/662,989, filed Jun. 21, 2024, titled INSERT CATHETER WITH PRE-SHAPED TIP, and U.S. Provisional Patent Application No. 63/664,675, filed Jun. 26, 2024, titled INSERT CATHETER WITH PRE-SHAPED TIP, the entire content of each of which is incorporated by reference herein for all purposes and forms as part of this specification.

### BACKGROUND

Stroke is the third most common cause of death in the United States and the most disabling neurologic disorder. Approximately 700,000 patients suffer from stroke annually. Stroke is a syndrome characterized by the acute onset of a neurological deficit that persists for at least 24 hours, reflecting focal involvement of the central nervous system, and is the result of a disturbance of the cerebral circulation. Its incidence increases with age. Risk factors for stroke include systolic or diastolic hypertension, hypercholesterolemia, cigarette smoking, heavy alcohol consumption, and oral contraceptive use.

Hemorrhagic stroke accounts for 20% of the annual stroke population. Hemorrhagic stroke often occurs due to a rupture of an aneurysm or arteriovenous malformation bleeding into the brain tissue, resulting in cerebral infarction. The remaining 80% of the stroke population are ischemic strokes and are caused by occluded vessels that deprive the brain of oxygen-carrying blood. Ischemic strokes are often caused by emboli or pieces of thrombotic tissue that have dislodged from other body sites or from the cerebral vessels themselves to occlude in the narrow cerebral arteries more distally. When a patient presents with neurological symptoms and signs which resolve completely within 1 hour, the term transient ischemic attack (TIA) is used. Etiologically, TIA and stroke share the same pathophysiologic mechanisms and thus represent a continuum based on persistence of symptoms and extent of ischemic insult.

Emboli occasionally form around the valves of the heart or in the left atrial appendage during periods of irregular heart rhythm and then are dislodged and follow the blood flow into the distal regions of the body. Those emboli can pass to the brain and cause an embolic stroke. As will be discussed below, many such occlusions occur in the middle cerebral artery (MCA), although such is not the only site where emboli come to rest.

When a patient presents with neurological deficit, a diagnostic hypothesis for the cause of stroke can be generated based on the patient's history, a review of stroke risk factors, and a neurologic examination. If an ischemic event is suspected, a clinician can tentatively assess whether the patient has a cardiogenic source of emboli, large artery extracranial or intracranial disease, small artery intraparenchymal disease, or a hematologic or other systemic disorder. A head CT scan is often performed to determine whether the patient has suffered an ischemic or hemorrhagic insult. Blood would be present on the CT scan in subarachnoid hemorrhage, intraparenchymal hematoma, or intraventricular hemorrhage.

Traditionally, emergent management of acute ischemic stroke consisted mainly of general supportive care, e.g. hydration, monitoring neurological status, blood pressure control, and/or anti-platelet or anti-coagulation therapy. In 1996, the Food and Drug Administration approved the use of Genentech Inc.'s thrombolytic drug, tissue plasminogen activator (t-PA) or Activase®, for treating acute stroke. A randomized, double-blind trial, the National Institute of Neurological Disorders and t-PA Stroke Study, revealed a statistically significant improvement in stoke scale scores at 24 hours in the group of patients receiving intravenous t-PA within 3 hours of the onset of an ischemic stroke. Since the approval of t-PA, an emergency room physician could, for the first time, offer a stroke patient an effective treatment besides supportive care.

However, treatment with systemic t-PA is associated with increased risk of intracerebral hemorrhage and other hemorrhagic complications. Patients treated with t-PA were more likely to sustain a symptomatic intracerebral hemorrhage during the first 36 hours of treatment. The frequency of symptomatic hemorrhage increases when t-PA is administered beyond 3 hours from the onset of a stroke. Besides the time constraint in using t-PA in acute ischemic stroke, other contraindications include the following: if the patient has had a previous stroke or serious head trauma in the preceding 3 months, if the patient has a systolic blood pressure above 185 mm Hg or diastolic blood pressure above 110 mmHg, if the patient requires aggressive treatment to reduce the blood pressure to the specified limits, if the patient is taking anticoagulants or has a propensity to hemorrhage, and/or if the patient has had a recent invasive surgical procedure. Therefore, only a small percentage of selected stroke patients are qualified to receive t-PA.

Obstructive emboli have also been mechanically removed from various sites in the vasculature for years. Mechanical therapies have involved capturing and removing the clot, dissolving the clot, disrupting and suctioning the clot, and/or creating a flow channel through the clot. One of the first mechanical devices developed for stroke treatment is the MERCI Retriever System (Concentric Medical, Redwood City, Calif.). A balloon-tipped guide catheter is used to access the internal carotid artery (ICA) from the femoral artery. A microcatheter is placed through the guide catheter and used to deliver the coil-tipped retriever across the clot and is then pulled back to deploy the retriever around the clot. The microcatheter and retriever are then pulled back, with the goal of pulling the clot, into the balloon guide catheter while the balloon is inflated and a syringe is connected to the balloon guide catheter to aspirate the guide catheter during clot retrieval. This device has had initially positive results as compared to thrombolytic therapy alone.

Other thrombectomy devices utilize expandable cages, baskets, or snares to capture and retrieve clot. Temporary stents, sometimes referred to as stentrievers or revascularization devices, are utilized to remove or retrieve clot as well as restore flow to the vessel. A series of devices using active laser or ultrasound energy to break up the clot have also been utilized. Other active energy devices have been used in conjunction with intra-arterial thrombolytic infusion to accelerate the dissolution of the thrombus. Many of these devices are used in conjunction with aspiration to aid in the removal of the clot and reduce the risk of emboli. Suctioning of the clot has also been used with single-lumen catheters and syringes or aspiration pumps, with or without adjunct disruption of the clot. Devices which apply powered fluid vortices in combination with suction have been utilized to improve the efficacy of this method of thrombectomy. Finally, balloons or stents have been used to create a patent lumen through the clot when clot removal or dissolution was not possible.

Notwithstanding the foregoing, there remains a need for new devices and methods for treating vasculature occlusions in the body, including acute ischemic stroke and occlusive cerebrovascular disease. In particular, as will be discussed in more detail below, because of the variation in levels of tortuosity and the large variability of certain segments of the intracranial carotid artery (e.g., the petrous-cavernous path) in stroke patients, there is a need for an anatomy matched catheter design.

### SUMMARY

There is provided in accordance with one aspect of the present disclosure a neurovascular catheter. The neurovascular catheter can include an elongate flexible body including a length of at least about 130 cm. The elongate flexible body including a distal portion including a pre-shaped tip and having a length between about 2 cm to 9 cm, a transitional portion proximal to the distal portion that is less flexible than the distal portion, wherein the transitional portion has a length of between about 10 cm and about 18 cm; and a proximal portion proximal to the transitional portion that is less flexible than the transitional portion, wherein the proximal portion has a length between about 110 cm and about 115 cm. A flexibility profile of the elongate flexible body can be measurable with a cantilever beam test with a 5 mm gage length and 4 mm displacement to determine a stiffness measured in peak load value/distance. The stiffness in the distal portion can be between about 10 gF/mm and about 200 gF/mm; the stiffness in the transitional portion can increase from between about 100 gF/mm and about 200 gF/mm to between about 800 gF/mm and about 1000 gF/mm over the length of the transitional portion; and the stiffness in the proximal portion can be between about 750 gF/mm and about 1150 gF/mm.

In some aspects, the elongate flexible body can include an inner liner, a braid wrapped around inner liner, and a jacket positioned radially outward from the inner line. The inner liner can extend and entire length of the elongate flexible body, and the braid can from about 6 cm to about 8 cm from a distal end of the elongate tubular body. In some cases, the jacket can include a plurality of tubular segments having a durometer that decreases in a distal direction.

In some aspects, the stiffness in a region spanning the distal portion and the transitional portion increases in a proximal direction from between about 125 gF/mm and about 135 gF/mm to between about 240 gF/mm and about 250 gF/mm over a distance of about 10 mm. In some cases, the stiffness in a region of the distal portion increases in a proximal direction from between about 75 gF/mm and about 85 gF/mm to between about 125 gF/mm and about 135 gF/mm over a distance of about 40 mm. The stiffness in a region of the distal portion can increase in a proximal direction from between about 75 gF/mm and about 85 gF/mm to between about 125 gF/mm and about 135 gF/mm over a distance of about 40 mm. In some aspects, the stiffness of the transitional portion increases in a proximal direction from between about 240 gF/mm and about 250 gF/mm to between about 850 gF/mm and about 860 gF/mm over a distance of about 12 cm.

In some aspects, an outer diameter of the elongate flexible body along the distal portion tapers in a distal direction from between about 0.075 in and about 0.085 in to between about 0.055 in and about 0.065 in. over a distance of between about 5.5 cm and about 6.5 cm.

There is also provided in accordance with one aspect of the present disclosure a neurovascular catheter for delivery to an ostium of the aortic arch. The neurovascular catheter can include an elongate flexible body including a length of at least about 130 cm. The elongate flexible body can include a distal portion including a pre-shaped tip, a transitional portion proximal to the distal portion, and a proximal portion proximal to the transitional; wherein the elongate flexible body has an outer diameter of about 6 F.

In some aspects, the pre-shaped tip includes: a first curve defining a first concave side and a first convex side; a second curve defining a second concave side and a second convex side; and a third curve defining a third concave side and a third convex side. In some cases a longitudinal axis of the elongate flexible body and a first section of the pre-shaped tip distal to the first curve can form an angle from about 120° to about 180°. The first section of the pre-shaped tip and a second section of the pre-shaped tip distal to the second curve can form an angle from about 10° to about 50°. In some cases, the second section of the pre-shaped tip and a third section of the pre-shaped tip distal to the third curve can form an angle from about 90° to about 140°. In some aspects, a distance between a distal end of the elongate flexible body and a longitudinal axis of the elongate flexible body along an axis perpendicular to the longitudinal axis can be between about 1 cm and about 6 cm.

In some aspects, the pre-shaped tip includes a curve defining a concave side and a convex side. A longitudinal axis of the elongate flexible body and a section of the pre-shaped tip distal to the curve can form an angle from about 30° to about 90°. A distance between a distal end of the elongate flexible body and a longitudinal axis of the elongate flexible body along an axis perpendicular to the longitudinal axis can be between about 0.1 cm and about 3 cm.

In some aspects, the pre-shaped tip includes a length from about 16 cm to about 20 cm.

In some aspects, the pre-shaped tip includes a first curve defining a first concave side and a first convex side; a second curve defining a second concave side and a second convex side; and a third curve defining a third concave side and a third convex side. A longitudinal axis of the elongate flexible body and a first section of the pre-shaped tip distal to the first curve can form an angle from about 40° to about 140°. In some cases, the first section of the pre-shaped tip and a second section of the pre-shaped tip distal to the second curve form an angle from about 10° to about 70°. The second section of the pre-shaped tip and a third section of the pre-shaped tip distal to the third curve can form an angle from about 100° to about 200°. In some aspects, a distance between a longitudinal axis of the elongate flexible body and an apex of the second convex side along an axis perpendicular to the longitudinal axis can be between about 1 cm and about 6 cm. A distance between a distal tip of the elongate flexible body and an apex of the second convex side along an axis perpendicular to the longitudinal axis can be between about 1 cm and about 5 cm.

In some aspects, the ostium of the aortic arch branches into of the right brachiocephalic artery, the left common carotid artery, and the left subclavian artery.

In some aspects, a stiffness of the elongate flexible body increases from a distal end of the elongate flexible body to a proximal end of the elongate flexible body.

### BRIEF DESCRIPTION OF THE DRAWINGS

FIG. 1 illustrates a side elevational schematic view of an example of an intracranial aspiration catheter with a distal segment in a proximally retracted configuration.

FIG. 2 illustrates a side elevational view of the intracranial aspiration catheter of FIG. 1, with the distal segment in a distally extended configuration.

FIG. 3 illustrates cerebral arterial vasculature including the Circle of Willis, and an access catheter positioned at an occlusion in the left carotid siphon artery.

FIGS. 4A-4E show a sequence of steps involved in positioning of the catheter and aspirating obstructive material from the middle cerebral artery and the subsequent removal of the catheter following aspiration of the obstructive material.

FIGS. 5A-5F depict a sequence of steps to access a neurovascular occlusion for aspiration.

FIGS. 6A-6F depicts an alternative sequence of steps involved in accessing a neurovascular occlusion for aspiration.

FIG. 7A illustrates a cross-sectional view of a catheter according to an embodiment.

FIG. 7B illustrates the catheter shown in FIG. 7A with a rapid exchange port.

FIG. 7C illustrates a portion of a catheter according to one embodiment with a braid wrapped around an inner liner.

FIG. 8A illustrates an example of an insert catheter with a first pre-shaped tip.

FIG. 8B illustrates the pre-shaped tip of the insert catheter shown in FIG. 8A.

FIG. 8C illustrates the insert catheter shown in FIG. 8A positioned inside the vasculature of the patient.

FIG. 9A illustrates an example of an insert catheter with a second pre-shaped tip.

FIG. 9B illustrates the pre-shaped tip of the insert catheter shown in FIG. 9A.

FIG. 9C illustrates the insert catheter shown in FIG. 9A positioned inside the vasculature of the patient.

FIG. 10A illustrates an example of an insert catheter with a third pre-shaped tip.

FIG. 10B illustrates the pre-shaped tip of the insert catheter shown in FIG. 10A.

FIG. 10C illustrates the insert catheter shown in FIG. 10A positioned inside the vasculature of the patient.

FIG. 10D illustrates additional interventional devices advanced over the insert catheter shown in FIG. 10C.

FIG. 11 illustrates a graph showing a flexibility profile of distal portion of an insert catheter disclosed herein.

### DETAILED DESCRIPTION

### Overview

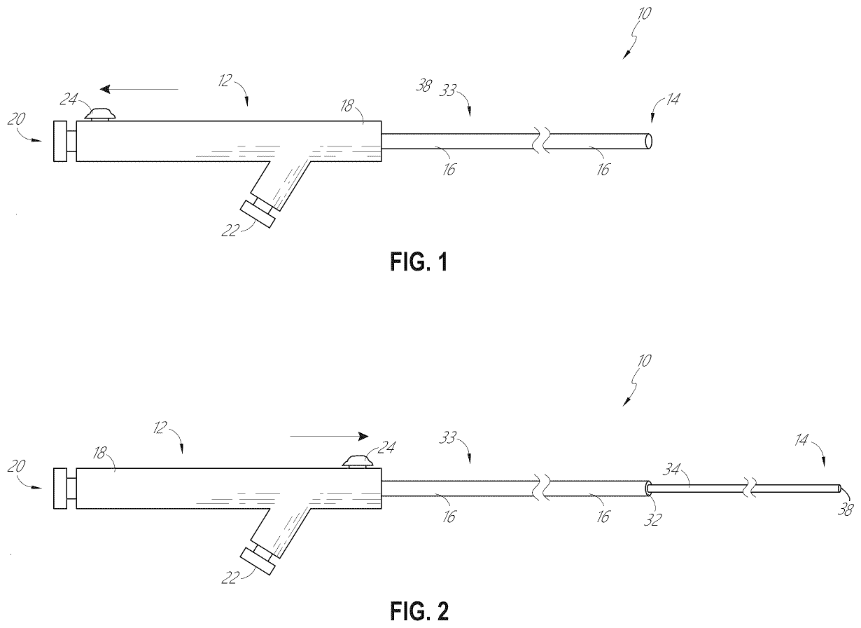

FIG. 1 illustrates an embodiment of a catheter 10 in accordance with one aspect of the present disclosure. Although primarily described in the context of an axially extendable distal segment aspiration catheter with a single central lumen, the presently disclosed catheter can readily be modified to incorporate additional structures, such as permanent or removable column strength enhancing mandrels, two or more lumen such as to permit drug, contrast or irrigant infusion or to supply inflation media to an inflatable balloon carried by the catheter, or combinations of these features, as will be readily apparent to one of skill in the art in view of the disclosure herein. In addition, the presently disclosed catheter will be described primarily in the context of removing obstructive material from remote vasculature in the brain, but has applicability as an access catheter for delivery and removal of any of a variety of diagnostics or therapeutic devices with or without aspiration.

The catheters disclosed herein may readily be adapted for use throughout the body wherever it may be desirable to distally advance a low profile distal catheter segment from a larger diameter proximal segment. For example, the presently disclosed axially extendable catheter shafts may be dimensioned for use throughout the coronary and peripheral vasculature, the gastrointestinal tract, the urethra, ureters, Fallopian tubes and other lumens and potential lumens, as well. The telescoping structure presently disclosed may also be used to provide minimally invasive percutaneous tissue access, such as for diagnostic or therapeutic access to a solid tissue target (e.g., breast or liver or brain biopsy or tissue excision), delivery of laparoscopic tools or access to bones such as the spine for delivery of screws, bone cement or other tools or implants. Examples of such catheters are illustrated in, for example, U.S. Pat. No. 10,183,145 to Yang, et al., and U.S. Pat. No. 10,835,272 to Yang, et al. the disclosure of which are incorporated in its entirety herein by reference.

As shown in FIG. 1, the catheter 10 generally comprises an elongate tubular body 16 extending between a proximal end 12 and a distal functional end 14. The length of the tubular body 16 depends upon the desired application. For example, lengths in the area of from about 120 cm to about 140 cm or more are typical for use in femoral access percutaneous transluminal coronary applications. Intracranial or other applications may call for a different catheter shaft length depending upon the vascular access site, as will be understood in the art. In some embodiments, the length of the tubular body 16 is limited by commercial products on the market that can be inserted through the catheter 10. As will be discussed in more detail below, in some embodiments, the length of the tubular body 16 can be at least about 100 cm, at least about 101 cm, at least about 102 cm, at least about 103 cm, at least about 104 cm, at least about 105 cm, at least about 106 cm, at least about 107 cm, at least about 108 cm, at least about 109 cm, at least about 110 cm, between about 100 cm to about 102 cm, between about 102 cm to about 104 cm, between about 104 cm to about 106 cm, between about 106 cm to about 108 cm, between about 108 cm to about 110 cm, less than about 110 cm, and any value in between the ranges listed, including endpoints. This length of the tubular body 16 is shorter and can provide for improved ergonomic hand placement. As well, the length of the tubular body 16 can reduce the excess length controlled by the physician which can help to improve the efficiency of the procedure.

In the illustrated embodiment, the tubular body 16 is divided into at least a fixed proximal section 33 and an axially extendable and retractable distal section 34 separated at a transition 32. FIG. 2 illustrates a side elevational view of the catheter 10 shown in FIG. 1, with the distal segment in a distally extended configuration. In some cases, however, the distal section 34 can include an additional catheter. For example, the distal section 34 can include a catheter, or a portion thereof, configured to extend and be advanced or retracted inside the tubular body 16. In some cases, the distal section 34 can have an outer diameter smaller than an inner diameter of the proximal section 33, thereby allowing the catheter having the distal section 34 to be advanced and/or retracted through the tubular body 16.

The inner diameter of the distal section 34 may be between about 0.030 inches and about 0.112 inches, between about 0.040 inches and about 0.102 inches, between about 0.045 inches and about 0.097 inches, between about 0.050 inches and about 0.092 inches, between about 0.055 inches and about 0.087 inches, between about 0.060 inches and about 0.082 inches, between about 0.062 inches and about 0.080 inches, between about 0.064 inches and about 0.078 inches, between about 0.066 inches and about 0.076 inches, between about 0.068 inches and about 0.074 inches, or between about 0.070 inches and about 0.072 inches.

The inner diameter and the outer diameter of the distal section 34 may be constant or substantially constant along its longitudinal length. Alternatively, the distal section 34 may be tapered near its distal end. The distal section 34 may be tapered at less than or equal to about 1 cm, about 2 cm, about 3 cm, about 4 cm, about 5 cm, about 7 cm, about 10 cm, about 15 cm, about 20 cm, about 23 cm, about 25 cm, about 30 cm, about 31 cm, about 35 cm, about 40 cm, about 45 cm, about 50 cm, about 60 cm, or about 70 cm from its distal end. In some embodiments, the taper may be positioned between about 25 cm and about 35 cm from the distal end of the distal section 34.

The inner diameter of the distal section 34 may be tapered or decreased in the distal direction near the distal end to an internal diameter that is less than or equal to about 95%, about 90%, about 85%, about 80%, about 75%, about 70%, about 65%, about 60%, about 55%, or about 50% of the adjacent, untapered internal diameter. In some embodiments, the internal diameter of the tapered distal section 34 may be between about 50% and about 70% of the adjacent, untapered internal diameter. For example, the untapered internal diameter at the proximal end of the distal section 34 may be about 0.071 inches and the tapered internal diameter at the distal end of the distal section 34 may be about 0.035 inches, 0.045 inches, or 0.055 inches. The inner diameter of the distal section 34 may be tapered or increased near the distal end by greater than or equal to about 102%, 104%, 106%, 108%, or more of the internal diameter just proximal to a transition into the taper. The tapered inner diameter of the distal section 34 may be less than or equal to about 0.11 inches, about 0.1 inches, about 0.090 inches, about 0.080 inches, about 0.070 inches, about 0.065 inches, about 0.060 inches, about 0.055 inches, about 0.050 inches, about 0.045 inches, about 0.040 inches, about 0.035 inches, about 0.030 inches, about 0.025 inches, about 0.020 inches, about 0.015 inches, or about 0.010 inches. In some embodiments, the length of the distal tapered portion of the distal section 34 may be between about 25 cm and about 35 cm, between about 25 cm and about 30 cm, between about 30 cm and 35 cm, or approximately 30 cm.

The length of the distal section 34 may be between about 13 cm and about 53 cm, between about 18 cm and about 48 cm, between about 23 cm and about 43 cm, between about 28 cm and about 38 cm, between about 29 cm and about 39 cm, between about 30 cm and about 40 cm, between about 31 cm and about 41 cm, or between about 32 cm and about 42 cm. The length of the distal section 34 may be less than or equal to about 20 cm, about 25 cm, about 30 cm, about 33 cm, about 35 cm, about 40 cm, about 41 cm, about 45 cm, about 50 cm, about 55 cm, about 60 cm, about 70 cm, or about 80 cm. The length of the distal section 34 may depend on the degree of tapering of the internal diameter of the distal section 34.

In cases where the distal section 34 is part of a separate catheter and/or medical instrument, the catheter including the distal section 34 can have a length such that the distal section 34 can extend between about 13 cm and about 53 cm, between about 18 cm and about 48 cm, between about 23 cm and about 43 cm, between about 28 cm and about 38 cm, between about 29 cm and about 39 cm, between about 30 cm and about 40 cm, between about 31 cm and about 41 cm, or between about 32 cm and about 42 cm, beyond a distal end of the tubular section 16 (e.g., the portion of the tubular body 16 where the transition 32 is located). The distal section 34 of the catheter may be less than or equal to about 20 cm, about 25 cm, about 30 cm, about 33 cm, about 35 cm, about 40 cm, about 41 cm, about 45 cm, about 50 cm, about 55 cm, about 60 cm, about 70 cm, or about 80 cm.

The proximal end 12 of catheter 10 can include a manifold 18 having one or more access ports. In some embodiments, manifold 18 is provided with a proximal port such as a guidewire port 20 in an over-the-wire construction, and at least one side port such as aspiration port 22. Alternatively, the aspiration port 22 may be omitted if the procedure involves removal of the guidewire proximally from the guidewire port 20 following placement of the aspiration catheter, and aspiration through the guidewire port. Additional access ports and lumen may be provided as needed, depending upon the functional capabilities of the catheter. Manifold 18 may be injection molded from any of a variety of medical grade plastics, or formed in accordance with other techniques known in the art.

In some embodiments, the manifold 18 may be provided with a control 24, for controlling the axial position of the distal segment 34 of the catheter. Control 24 may take any of a variety of forms depending upon the mechanical structure and desired axial range of travel of the distal segment 34. In the illustrated embodiment, control 24 includes a slider switch which is mechanically axially movably linked to the distal segment such that proximal retraction of the slider switch 24 produces a proximal movement of the distal segment 34. This retracts the distal segment 34 into the proximal section 33 as illustrated in FIG. 1. Distal axial advancement of the slider switch 24 produces a distal axial advance of the distal segment 34, as illustrated in FIG. 2.

Any of a variety of controls may be utilized, including switches, buttons, levers, rotatable knobs, pull/push wires, and others which will be apparent to those of skill in the art in view of the disclosure herein. The control will generally be linked to the distal segment by a control wire 42.

In some embodiments, the proximal section 33 and distal section 34 maybe provided as separate devices, in which construction the proximal control may be omitted. The distal end of proximal section 33 may be provided with one or more jaws, for morcellating or otherwise breaking thrombus or other obstruction into pieces or otherwise facilitating aspiration. The proximal section 33 may additionally be mechanically coupled to or adapted for coupling to a source of vibrational or rotational movement, such as to provide the intermittent or pulsatile movement discussed elsewhere herein to facilitate navigation into the vasculature. Using axial reciprocation, and/or rotation, and/or biting action of the distal jaws, the clinician may be able to reach the obstruction using proximal section 33.

### Overview of Method of Treatment

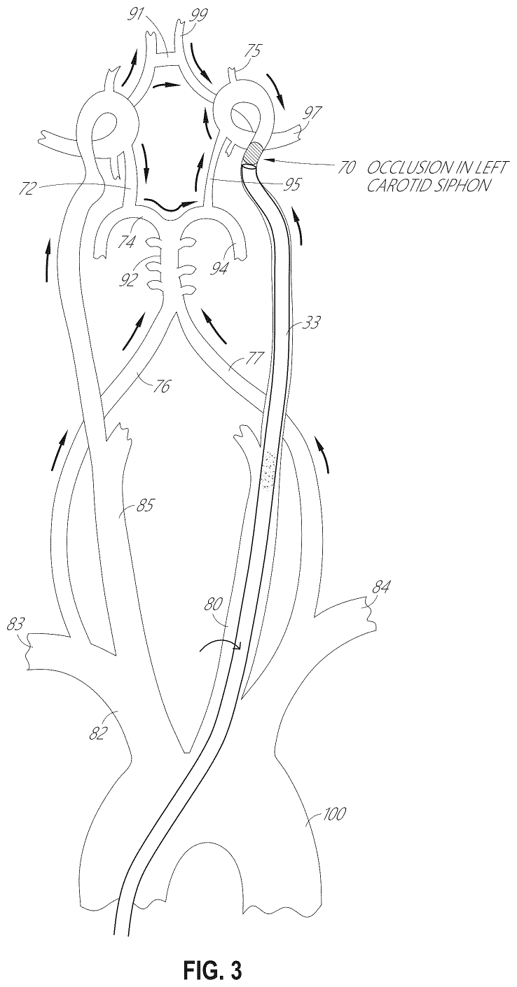

In some embodiments, the proximal section 33 of the catheter 10 is able to reach an obstruction in the left carotid siphon. FIG. 3 illustrates a cerebral arterial vasculature including the Circle of Willis, and an access catheter positioned at an occlusion in the left carotid siphon artery. In some examples, if the proximal section 33 is not able to advance sufficiently close to the obstruction, a separate telescoping distal section 34 may be introduced into the proximal section 33 and advanced therethrough and beyond, as illustrated in FIGS. 2 and 4-6F, to reach the obstruction. In some cases, the proximal section 33 of the catheter 10 may correspond to a guide catheter, and the distal section 34 may correspond to a separate insert catheter, such as insert catheters 1124, 1300, 1400, 1500, and/or 1600, which are further described herein. In such cases, the insert catheter may be advanced and/or retracted through the guide catheter.

The cerebral circulation is regulated in such a way that a constant total cerebral blood flow (CBF) is generally maintained under varying conditions. For example, a reduction in flow to one part of the brain, such as in acute ischemic stroke, may be compensated by an increase in flow to another part, so that CBF to any one region of the brain remains unchanged. More importantly, when one part of the brain becomes ischemic due to a vascular occlusion, the brain compensates by increasing blood flow to the ischemic area through its collateral circulation.

### Treatment of Acute Occlusion

FIG. 3 depicts cerebral arterial vasculature including the Circle of Willis. Aorta 100 gives rise to right brachiocephalic artery 82, left common carotid artery (CCA) 80, and left subclavian artery 84. The brachiocephalic artery 82 further branches into right common carotid artery 85 and right subclavian artery 83. The left CCA gives rise to left internal carotid artery (ICA) 90 which becomes left middle cerebral artery (MCA) 97 and left anterior cerebral artery (ACA) 99. Anteriorly, the Circle of Willis is formed by the internal carotid arteries, the anterior cerebral arteries, and anterior communicating artery 91 which connects the two ACAs. The right and left ICA also send right posterior communicating artery 72 and left posterior communicating artery 95 to connect, respectively, with right posterior cerebral artery (PCA) 74 and left PCA 94. The two posterior communicating arteries and PCAs, and the origin of the posterior cerebral artery from basilar artery 92 complete the circle posteriorly.

When an occlusion occurs acutely, for example, in left carotid siphon 70, as depicted in FIG. 3, blood flow in the right cerebral arteries, left external carotid artery 78, right vertebral artery 76 and left vertebral artery 77 increases, resulting in directional change of flow through the Circle of Willis to compensate for the sudden decrease of blood flow in the left carotid siphon. Specifically, blood flow reverses in right posterior communicating artery 72, right PCA 74, left posterior communicating artery 95. Anterior communicating artery 91 opens, reversing flow in left ACA 99, and flow increases in the left external carotid artery, reversing flow along left ophthalmic artery 75, all of which contribute to flow in left ICA 90 distal the occlusion to provide perfusion to the ischemic area distal to the occlusion.

As illustrated in FIG. 3, the proximal segment of catheter 10 is transluminally navigated along or over the guidewire, to the proximal side of the occlusion. Transluminal navigation may be accomplished with the distal section 34 of the catheter in the first, proximally retracted configuration. This enables distal advance of the proximal section 33 until further progress is inhibited by small and/or tortuous vasculature. Alternatively, the distal section 34 is a separate device, and is not inserted into the proximal section 33 until it is determined that the proximal section 33 cannot safely reach the occlusion. In the example illustrated in FIG. 3, the occlusion may be safely reached by the proximal section 33, without the need to insert or distally extend a distal section 34.

The distal end of the proximal section 33 of aspiration catheter 10 is inserted typically through an incision on a peripheral artery over a guidewire and advanced as far as deemed safe into a more distal carotid or intracranial artery, such as the cervical carotid, terminal ICA, carotid siphon, MCA, or ACA. The occlusion site can be localized with cerebral angiogram or IVUS. In emergency situations, the catheter can be inserted directly into the symptomatic carotid artery after localization of the occlusion with the assistance of IVUS or standard carotid doppler and TCD.

If it does not appear that sufficient distal navigation of the proximal section 33 to reach the occlusion can be safely accomplished, the distal section 34 is inserted into the proximal port 20 and/or distally extended beyond proximal section 33 until the distal tip 38 is positioned in the vicinity of the proximal edge of the obstruction.

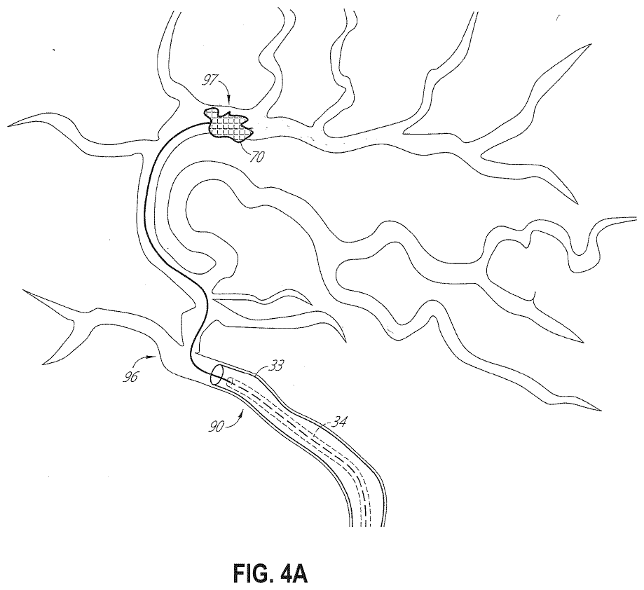

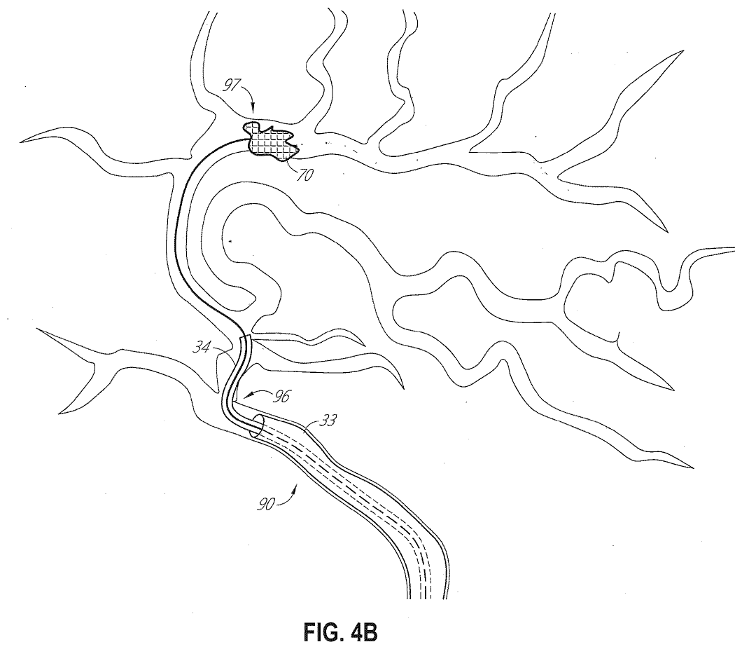

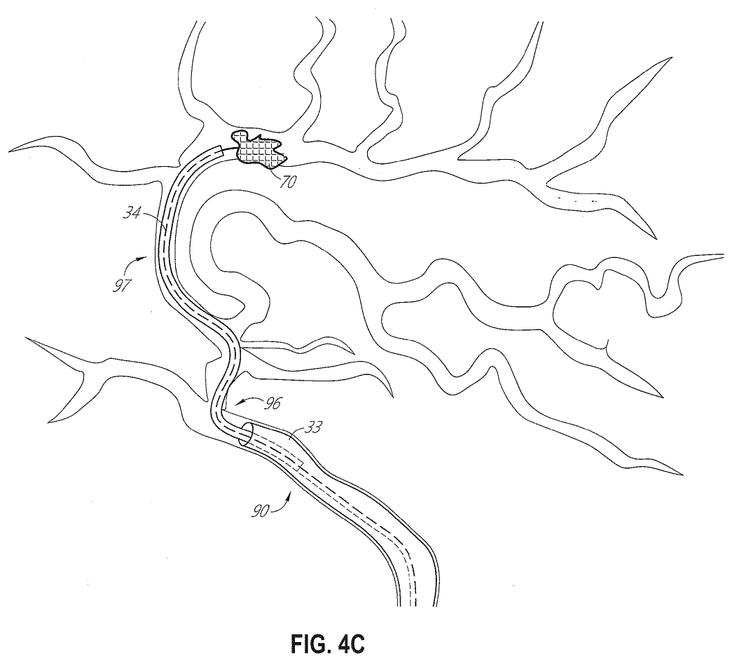

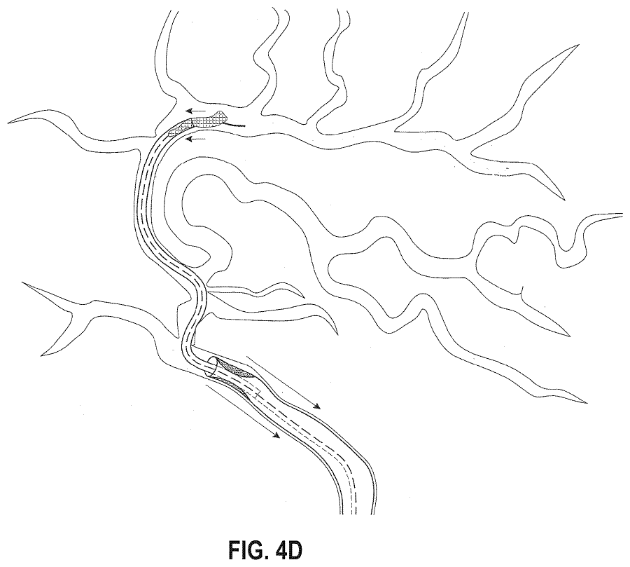

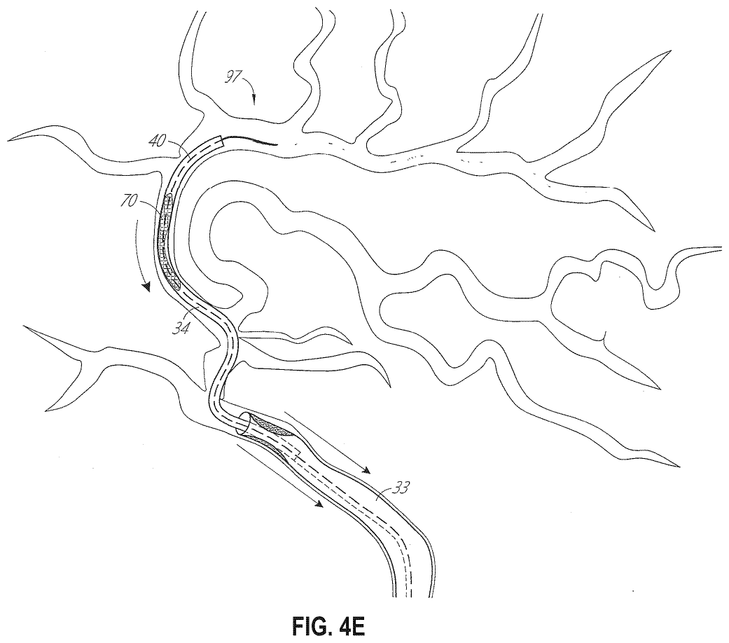

Referring to FIG. 4A, an obstruction 70 is lodged in the middle cerebral artery 97. Proximal section 33 is positioned in the ICA and not able to navigate beyond a certain point such as at the branch 96 to the MCA artery 97. The proximal section 33 may be provided with a distal section 34 carried therein. Alternatively, a separate distal section 34 may be introduced into the proximal end of proximal section 33 once the determination has been made that the obstruction 70 cannot be reached directly by proximal section 33 alone. As seen in FIGS. 4B and 4C, the distal section 34 may thereafter be transluminally navigated through the distal tortuous vasculature between proximal section 33 and the obstruction 70.

As shown in FIG. 4D, the obstruction 70 may thereafter be drawn into distal section 34. In some embodiments, the obstruction 70 can be drawn into the distal section 34 upon application of constant or pulsatile negative pressure. In some examples, the distal end of the distal section 34 can include jaws or other mechanical features for with or without the use of jaws or other activation on the distal end of distal section 34. FIG. 4E illustrates that once the obstruction 70 has either been drawn into distal section 34, or drawn sufficiently into distal section 34, the proximal section 33 and distal section 34 are thereafter proximally withdrawn.

### Treatment of Occlusion in Internal Carotid Artery

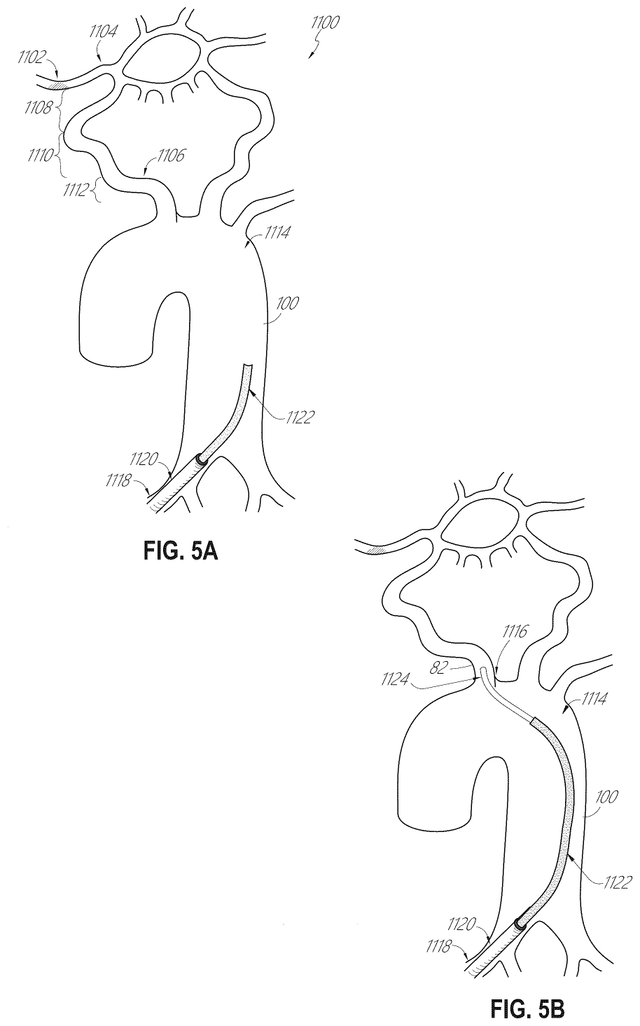

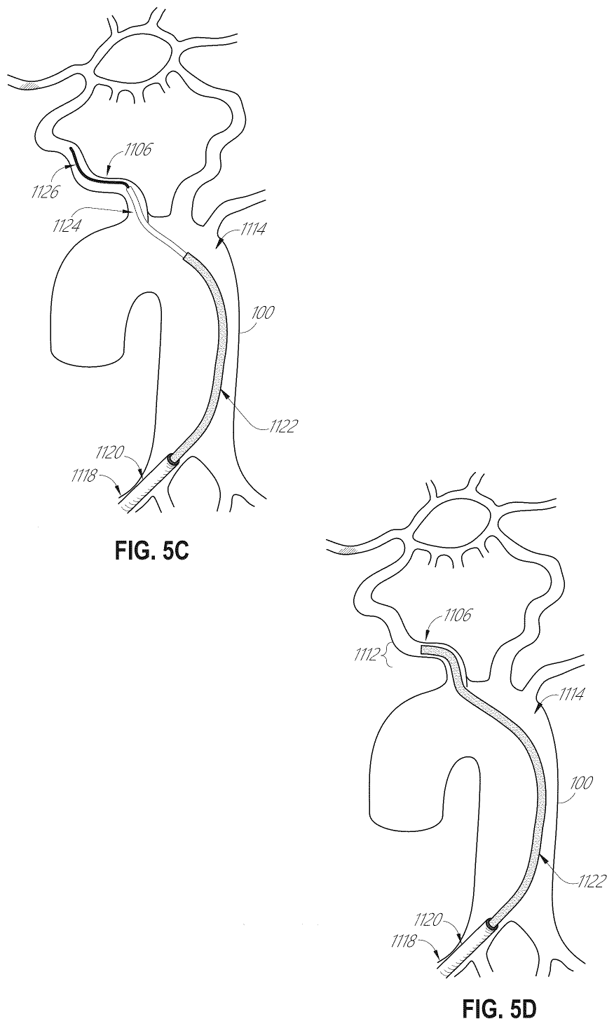

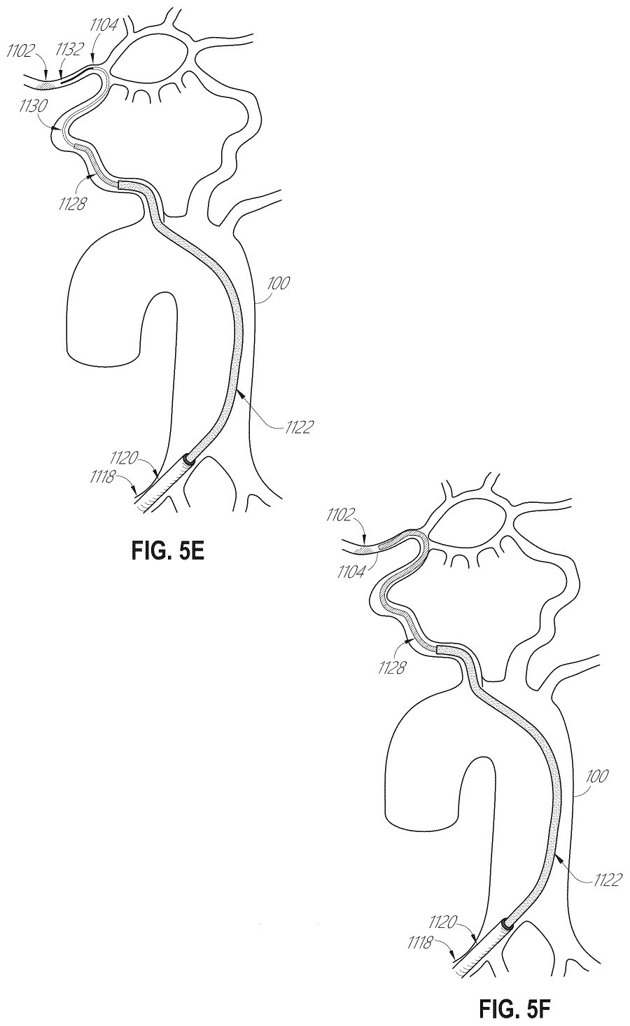

FIGS. 5A-5F illustrates accessing an occlusion positioned in the right middle cerebral artery of the right internal carotid artery. As shown, the cerebral circulation 1100 is simplified for the ease of demonstrating procedural steps. A thrombotic occlusion 1102 is in the right middle cerebral artery (RMCA) 1104. The RMCA 1104 branches from the right internal carotid artery (RICA) 1106. The RICA 1106 branches from the right common carotid artery (RCCA) (not shown). The RICA 1106 comprises cerebral 1108 (most distal from the aorta 100), cavernous 1110, and petrous 1112 (most proximal from the aorta 100) segments. The RCCA branches from the brachiocephalic artery. The brachiocephalic artery branches from the arch 1114 of the aorta 100.

The procedural steps for aspirating a thrombotic occlusion are described as follows. Referring to FIG. 5A, an introducer sheath 1120 is introduced at the femoral artery 1118. The outer diameter of the introducer sheath 1120 may be equal to or less than about 12F, 11 F, 10F, 9 F, 8F, 7 F, or 6 F. Then, a guide sheath 1122 is inserted through the introducer sheath 1120. The outer diameter of the guide sheath 1122 may be equal to or less than about 9F, 8 F, 7F, 6 F, 5F, 4 F, or 3 F, and the inner diameter of the introducer sheath 1120 may be greater than the outer diameter of the guide sheath 1122.

Referring to FIG. 5B, an insert catheter 1124 is inserted through the guide sheath 1122. The outer diameter of the insert catheter 1124 may be equal to or less than about 9F, 8 F, 7F, 6 F, 5F, 4 F, or 3 F, and the inner diameter of the guide sheath 1122 may be greater than the outer diameter of the insert catheter 1124. In some cases, a first guidewire 1126 may be introduced through the insert catheter 1124 (not shown in FIG. 5B). Then, the guide sheath 1122, the insert catheter 1124, and optionally the first guidewire 1126 are tracked up to the aortic arch 1114. The insert catheter 1124 is used to engage the origin of a vessel. In FIG. 5B, the insert catheter 1124 engages the origin 1116 of the brachiocephalic artery 82. An angiographic run is performed by injecting contrast media through the insert catheter 1124. In the cases where the first guidewire 1126 is used before the angiographic run, the first guidewire 1126 is removed prior to injecting the contrast media.

Referring to FIG. 5C, the first guidewire 1126 is inserted through the lumen of the insert catheter 1124. Then, the first guidewire 1126, the insert catheter 1124, and the guide sheath 1122 are advanced together to the RICA 1106. Referring to FIG. 5D, due to the stiffness of a typical guide sheath 1122 currently available in the market (e.g., Neuron MAX System produced by Penumbra Inc.), the most distal vessel that the guide sheath 1122 could navigate to is the petrous segment 1112 of the RICA 1106. Once the first guidewire 1126, the insert catheter 1124, and the guide sheath 1122 are advanced to the RICA 1106, both the first guidewire 1126 and the insert catheter 1124 are removed.

Referring to FIG. 5E, a second guidewire 1132 loaded inside the central lumen of a reperfusion catheter 1130 (e.g., 3Max), which is loaded inside the central lumen of an aspiration catheter 1128 (e.g., ACE 68), are introduced through the guide sheath 1122. The diameter of the second guidewire 1132 may be equal to or less than about 0.03″, about 0.025″, about 0.02″, about 0.016″, about 0.014″, about 0.01″, or about 0.005″. The inner diameter of the reperfusion catheter 1130 may be greater than the outer diameter of the second guidewire 1132. The inner diameter of the aspiration catheter 1128 may be greater than the outer diameter of the reperfusion catheter 1130. The inner diameter of the guide sheath 1122 may be greater than the outer diameter of the aspiration catheter 1128. Then, the second guidewire 1132 is advanced distally and positioned at the proximal end of the clot 1102 in the MCA 1104.

Referring to FIG. 5F, the aspiration catheter 1128 is tracked over the reperfusion catheter 1130 and the second guidewire 1132 to the proximal end of the clot 1102 in the MCA 1104. Both the second guidewire 1132 and the reperfusion catheter 1130 are removed. A vacuum pressure is then applied at the proximal end of the aspiration catheter 1128 to aspirate the clot 1102 through the central lumen of the aspiration catheter 1128.

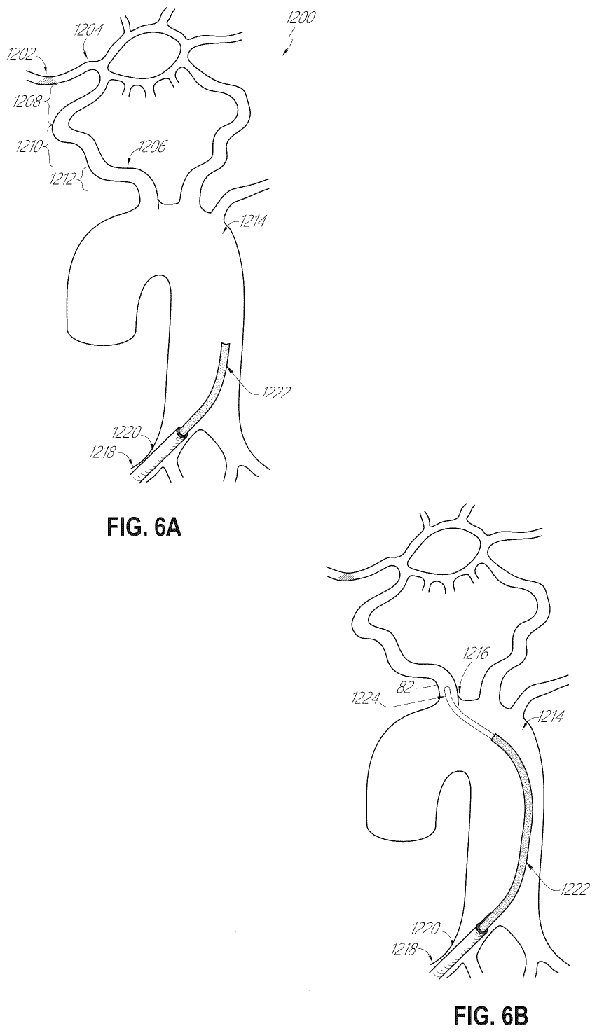

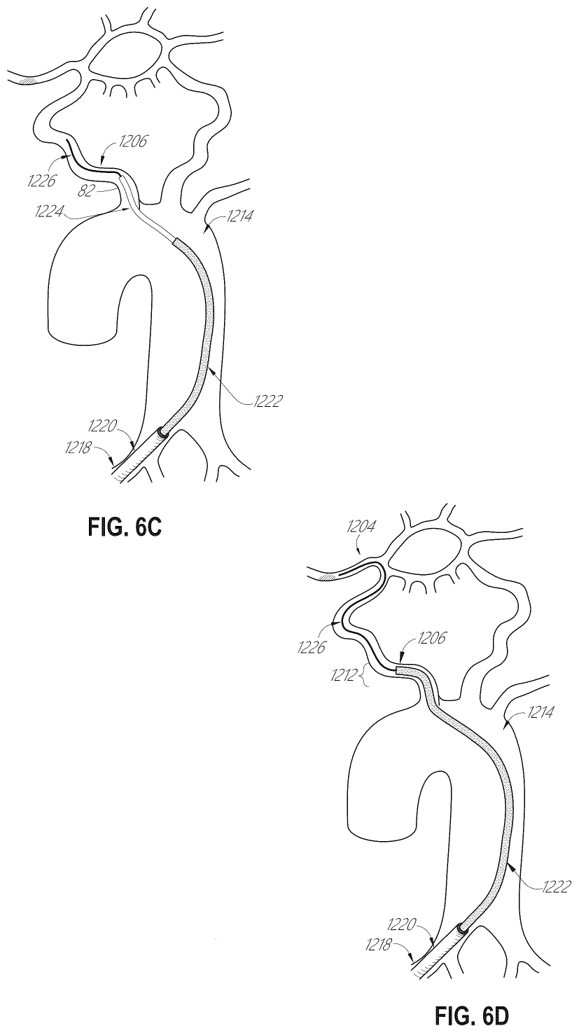

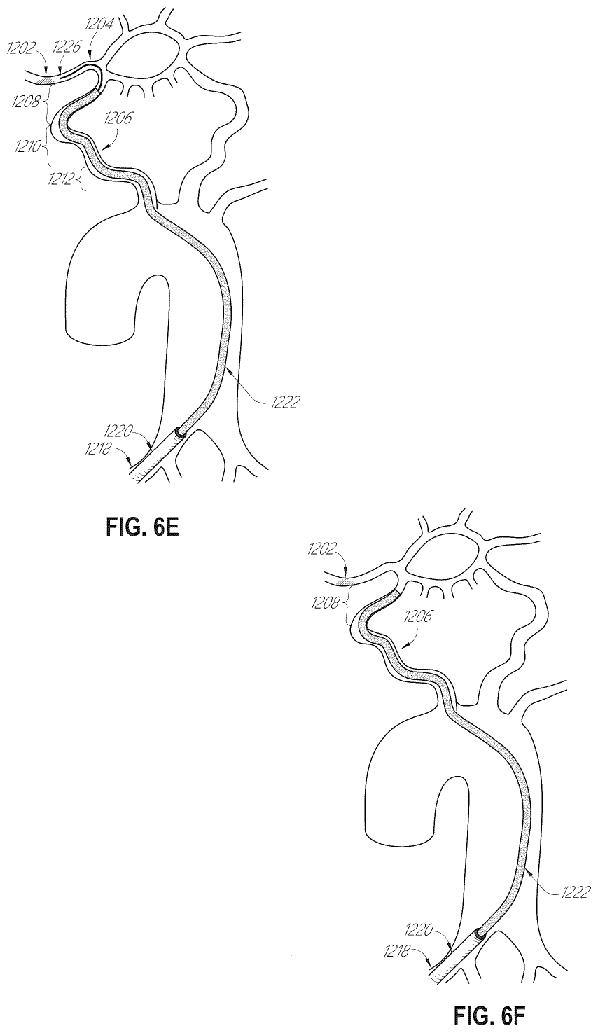

FIGS. 6A-6F illustrate an alternative and simplified method for aspirating a thrombotic occlusion. The alternative steps for aspirating a thrombotic occlusion make use of a transitional guidewire and a transitional guide sheath. The transitional guidewire has a soft and trackable distal segment with a smaller diameter so that the transitional guidewire may be advanced deeper than the guidewire 1126 described in FIG. 5C. In addition, the transitional guide sheath has a soft and trackable distal segment such that the transitional guide sheath may be advanced deeper than the guide sheath 1122 described in FIG. 5D. Using a transitional guidewire and a transitional guide sheath that can be advanced to an area near the clot eliminates the need to use a second guidewire or a reperfusion catheter to reach the clot.

Referring to FIG. 6A, an introducer sheath 1220 is introduced at the femoral artery 1218. The outer diameter of the introducer sheath 1220 may be equal to or less than about 12F, 11 F, 1° F., 9F, 8 F, 7 F, or 6 F. Then, a transitional guide sheath 1222 such as the combination access and aspiration catheter discussed in greater detail below is inserted through the introducer sheath 1120 at the femoral artery 1218. The outer diameter of the guide sheath 1222 may be equal to or less than about 9F, 8 F, 7F, 6 F, 5F, 4 F, or 3 F. Referring to FIG. 6B, an insert catheter 1224 is inserted through the transitional guide sheath 1222. The outer diameter of the insert catheter 1224 may be less than about 9F, 8 F, 7F, 6 F, 5F, 4 F, or 3 F, and the inner diameter of the transitional guide sheath 1222 may be greater than the outer diameter of the insert catheter 1224. In some cases, a first guidewire may be introduced through the insert catheter 1224 (not shown in FIG. 6B). The diameter of the proximal section of the first guidewire may be equal to or less than about 0.079″, about 0.066″, about 0.053″, about 0.038″, about 0.035″, about 0.030″, or about 0.013″.

The transitional guide sheath 1222, the insert catheter 1224, and optionally the first guidewire are tracked up to the aortic arch 1214. See FIG. 6B. The insert catheter 1224 may be used to select the origin of a vessel. In FIG. 6B, the insert catheter 1224 engages the origin 1216 of the brachiocephalic artery 82. An angiographic run may be performed by injecting contrast media through the insert catheter 1224. In the cases where the first guidewire is used before the angiographic run, the first guidewire is preferably removed prior to injecting the contrast media.

Referring to FIG. 6C, the transitional guidewire 1226 is inserted through the lumen of the insert catheter 1224 or guide sheath 1222. The diameter of at least a portion of the transitional guidewire 1226 (e.g., proximal diameter) is substantially similar to that of the first guidewire 1126. The diameter of at least a portion of the transitional guidewire 1226 (e.g., distal diameter) may be smaller than that of the first guidewire 1126 and may have a diameter along a proximal segment of at least about 0.030″ and in one implementation about 0.038″. A transition begins within the range of from about 15 cm-30 cm from the distal end, and typically no more than about 20 cm or 25 cm from the distal end, distally of which the diameter tapers down to no more than about 0.018″ and in one implementation about 0.016″. Referring to FIG. 6D, if utilized, the insert catheter 1224 may be removed because it is too stiff to be advanced to the MCA 1204. In some embodiments, the transitional guidewire 1226 provides sufficient back up support that the combination access and aspiration catheter 1224 may be advanced directly over the transitional guidewire without any intervening devices. Then, the transitional guidewire 1226 is advanced to the MCA 1204. The transitional guidewire 1226 has a distal segment that has a smaller diameter than that of the first guidewire 1126 described in FIG. 5C. The distal segment of the transitional guidewire 1226 comprises a soft and atraumatic tip and can be tracked to the remote neurovasculature such as the MCA 1204, which is distal to the petrous segment 1212 of the ICA 1206.

Referring to FIG. 6E, the transitional guide sheath 1222 is advanced to or beyond the cavernous segment 1210 or the cerebral 1208 segment of the ICA 1206. Unlike the guide sheath 1122 described in FIG. 5D, the transitional guide sheath 1222 may be advanced to the cavernous segment 1210 or the cerebral 1208 segment of the ICA 1206 beyond the petrous segment 1212 because the transitional guide sheath 1222 has a soft yet trackable distal segment. The larger proximal diameter and stiffer body of the transitional guidewire 1226 may provide better support for the transitional guide sheath 1222 to track through the vasculature.

Referring to FIG. 6F, after the transitional guide sheath 1222 is advanced to the cerebral segment 1208 of the ICA 1206, the transitional guidewire 1226 is removed. Then, a vacuum pressure is applied at the proximal end of the transitional guide sheath 1222 to aspirate the clot 1202 through the central lumen of the transitional guide sheath 1222. The inner diameter of the transitional guide sheath 1222 may be equal to about or greater than about 0.100″, about 0.088″, about 0.080″, about 0.070″, or about 0.060″. The inner diameter of the transitional guide sheath 1222 is larger than the aspiration catheter 1128 described in FIG. 5E, which translates to more effective aspiration. The cross-sectional area of the central lumen of the transitional guide sheath 1222 may be almost twice as large as that of the largest aspiration catheter 1128 currently available.

If the guide sheath 1222 is not able to track deep enough into the distal vasculature to reach the clot or other desired target site, a telescopic extension segment as discussed elsewhere herein may be introduced into the proximal end of sheath 1222 and advanced distally to extend beyond the distal end of the sheath 1222 and thereby extend the reach of the aspiration system. In some embodiments, the extension segment has an ID of about 0.070″.

If thrombotic material is not able to be drawn into the sheath 1222 or extension segment under constant vacuum, pulsatile vacuum may be applied. If pulsatile vacuum does not satisfactorily capture the clot, an agitator may be advanced through the sheath 1222 and extension segment to facilitate drawing the clot into the central lumen.

### Insert Catheters

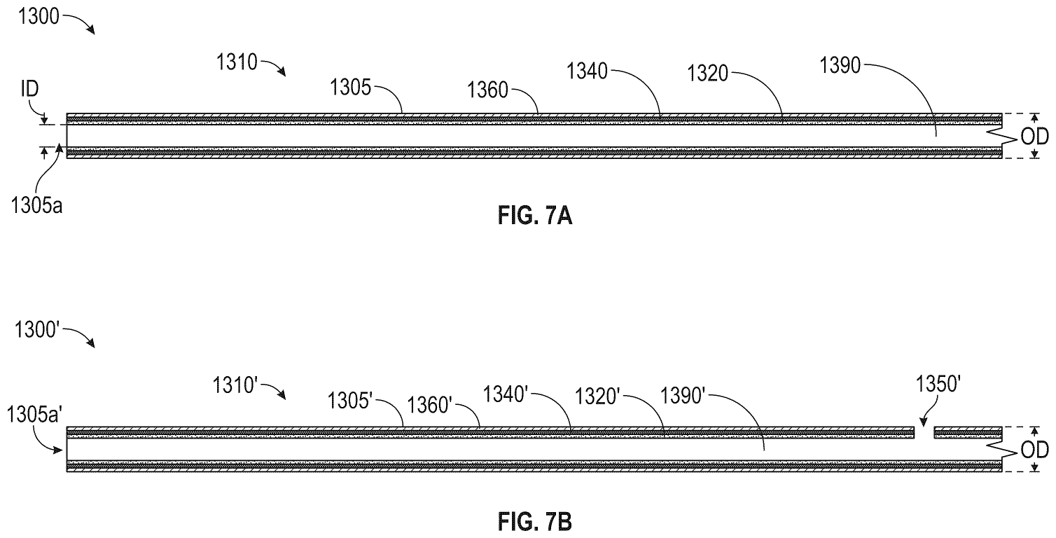

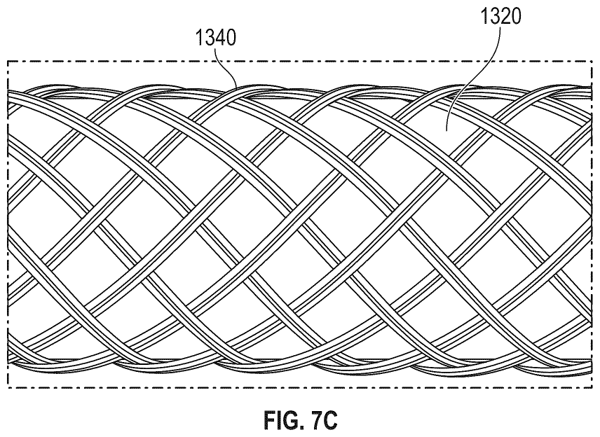

Any of the catheters disclosed herein may be provided with an inner liner, a reinforcing element, and/or an outer jacket. Referring to FIG. 7A, a distal portion 1310 of an insert catheter 1300 having a tubular body 1305 including a distal end 1305a, an inner liner 1320, a reinforcing element 1340, and an outer jacket 1360. In some cases, the reinforcing element 1340 can include a braid and/or a spring coil, such as the braid 1340 as shown in FIG. 7C. The reinforcing element 1340 can be positioned radially outward of the inner liner 1320. In some cases, the reinforcing element 1340 can also be embedded in the outer jacket 1360, which can be positioned radially outward of the inner liner 1320.

The insert catheter 1300 can also include a lumen 1390 extending radially inward from the inner liner 1320. The lumen 1390 can extend an entire length of the insert catheter 1300. The lumen 1390 can beneficially receive other interventional devices, such as a guidewire. In some cases, the distal portion 1310 may be radiopaque, which can beneficially allow visualization of the insert catheter 1300 under fluoroscopy.

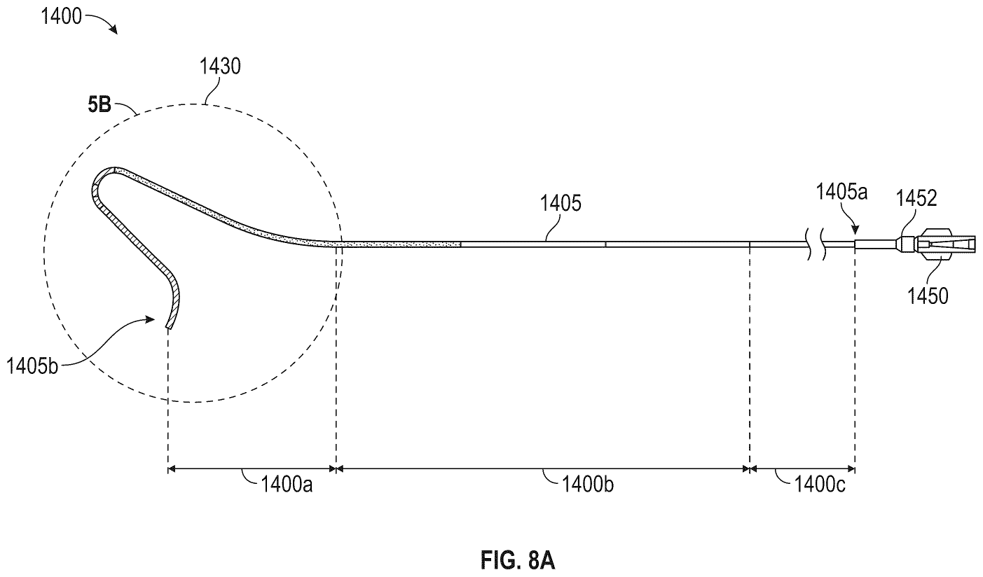

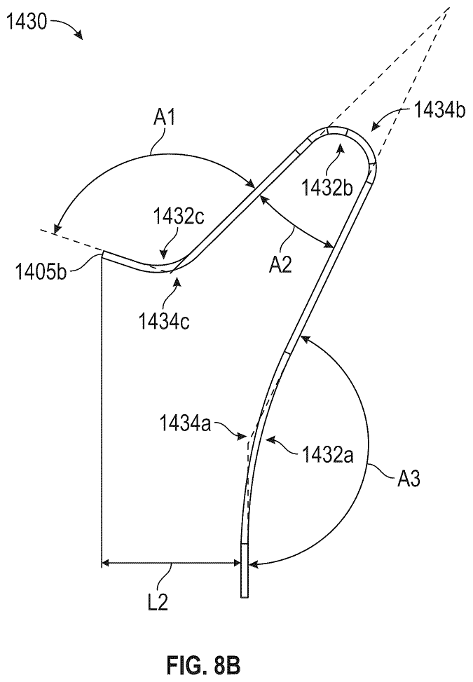

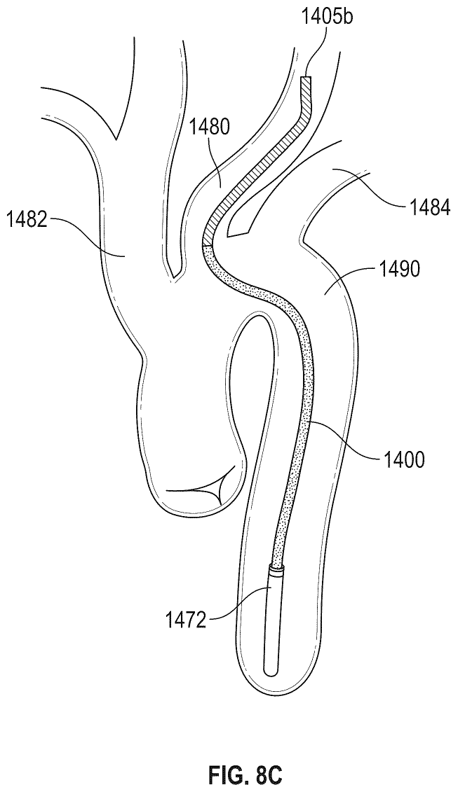

The reinforcing element 1340 and/or the outer jacket 1360 may extend an entire length of the inner liner 1320 along the distal portion 1310. In some cases, however, the reinforcing element 1340 and/or the outer jacket 1360 may extend less than entire length of the inner liner 1320 along the distal portion 1310. For example, the reinforcing element 1340 can extend distally along the distal portion 1310 and end about 10 mm, 15 mm, 20 mm, 40 mm, 50 mm, 60 mm, 70 mm, 75 mm, 80 mm, 85 mm, 90 mm, 100 mm, 120 mm, and/or 150 mm from the distal end 1305a. When the reinforcement element 1340 does not extend an entire length of the inner liner 1320, the distal-most portion of the insert catheter 1300 does not include a reinforcing element 1340, as will be described in greater details with reference to FIGS. 8A-10B.

The outer jacket 1360 may include a plurality of tubular segments positioned coaxially over the inner liner 1320 and/or the reinforcing element 1340. In some cases, the outer jacket 1360 can include at least two tubular segments, and in some cases 3, 4, 5, 6, 7, 8, 9, and/or 10 tubular segment. The plurality of tubular segments of the outer jacket 1360 can include durometers that decrease in a distal direction. The durometers of the plurality of tubular segments can range from about 35D to about 75D. The plurality of tubular segments of the outer jacket 1360 can have stiffness that decreases (that is, increasingly more flexible) in a distal direction, such as described further below with reference to FIG. 11.

In some cases, the insert catheter 1300 can include an outer diameter OD ranging from about 0.020 in. to about 0.12 in. For example, the outer diameter OD of the insert catheter 1300 can be from about 0.030 in. to about 0.11 in., from about 0.040 in. to about 0.10 in., from about 0.050 in. to about 0.09 in., from about 0.060 in. to about 0.090 in., or from about 0.070 in. to about 0.080 in. The outer diameter OD of the insert catheter 1300 may be constant through an entire length of the insert catheter 1300. In some cases, however, the outer diameter OD of the insert catheter 1300 may vary along different portions of the insert catheter 1300. As further described in relation to the insert catheters shown in FIGS. 8A-10B, a distal-most portion of the insert catheter 1300 may taper in a distal direction (that is, increasingly smaller towards the distal end 1305a). That is, the outer diameter OD of the insert catheter 1300 on the distal end 1305a may be smaller than the outer diameter OD along portions of the insert catheter 1300 proximal to the distal end 1305a.

The insert catheter 1300 can include an inner diameter ID (corresponding to the diameter of the lumen 1390) ranging from about 0.010 in. to about 0.070 in. For example, the inner diameter ID of the insert catheter 1300 can be from about 0.015 in. to about 0.065 in., from about 0.020 in. to about 0.060 in., from about 0.025 in. to about 0.055 in., from about 0.030 in. to about 0.050 in., and/or from about 0.035 in. to about 0.045 in. The inner diameter ID of the insert catheter 1300 may be constant through an entire length of the insert catheter 1300.

The insert catheter 1300 can have a length ranging from about 100 cm to about 170 cm. In some cases, the length of the insert catheter 1300 can be from about 110 cm. to about 160 cm., from about 120 cm. to about 150 cm., from about 130 cm, to about 145 cm, and/or from about 135 cm. to about 145 cm. The insert catheter 1300 can be longer than a guide catheter and/or sheath which the insert catheter 1300 can extend through.

As further described in relation to FIGS. 8A-10B, the insert catheter 1300 may include a pre-shaped tip. For example, the pre-shaped tip of the insert catheter 1300 may include one or more concave and/or convex portions. In some cases, at least portions of the pre-shaped tip can establish an angle relative to a longitudinal axis of the insert catheter 1390. The pre-shaped tip can beneficially allow for easier and faster access, insertion, and/or steerability of the insert catheter along tortuous and/or elongated vessels such as the aortic arch vessels. For example, the insert catheter 1300 can be advanced at least as far as the aortic arch, ostia of the aortic arch, and/or further into the vasculature (e.g., the right carotid artery).

In some cases, a stiffness rating of the insert catheter 1300 may vary along the length of the insert catheter 1300. For example, distal portions of the insert catheter 1300 may be softer than more proximal portions of the insert catheter 1300. Having a softer distal portion 1310 can beneficially prevent the insert catheter from damaging vessel walls when, for example, the insert catheter 1300 is advanced through the vasculature. The stiffer proximal portions, however, provide support which can beneficially facilitate pushability and/or allow the insert catheter 1300 to stay in place when not actively being advanced or retracted.

It may be desirable for the insert catheter to have a minimum threshold torque response. The minimum threshold torque response can allow rotational movement of the distal portion 1310 of the insert catheter 1300 to more closely replicate rotation applied to a proximal portion of the insert catheter 1300. This can beneficially allow for more predictable steerability and positioning of the insert catheter 1300 within the vasculature of a patient. For example, in some cases, the catheter 1300 can have an average torque response from about 0.001 Ncm/deg to about 0.005 Ncm/deg. In some cases, the catheter 1300 can have an average torque response from about 0.0012 Ncm/deg to about 0.0048 Ncm/deg, from about 0.002 Ncm/deg to about 0.0044 Ncm/deg, and/or from about 0.003 Ncm/deg to about 0.004 Ncm/deg. In some cases, the catheter 1300 can have a torque response from about 0.01 Ncm/deg to about 1.5 Ncm/deg. For example, the catheter 1300 can have a torque response from about 0.05 Ncm/deg to about 1.2 Ncm/deg, from about 0.1 Ncm/deg to about 0.9 Ncm/deg, and/or from about 0.3 Ncm/deg to about 0.7 Ncm/deg.

In some cases, the insert catheter 1300 can be used to deliver contrast media, saline, drugs, and/or other types of fluids into the vasculature of a patient. In some cases, the insert catheter 1300 can inject fluids at a pressure of up to 1500 PSI. For example, the insert catheter 1300 can inject fluids at a pressure of about 1400 PSI, 1300 PSI, 1200 PSI, 1100 PSI, 900 PSI, 800 PSI. In some cases, the insert catheter 1300 can inject fluids at a flow rate of at least about 5 mL/s. For the insert catheter 1300 can inject fluids at a flow rate of about 5 mL/s, 8 mL/s, 10 mL/s, 15 mL/s, 20 mL/s, 30 mL/s, 50 mL/s, 100 mL/s, etc.

Any of the insert catheters disclosed herein can be rapid exchange catheters. Rapid exchange catheters can include an exit port positioned distal to a proximal end of the catheter. For example, an insert catheter 1300′ can include an exit port 1350′, as shown in FIG. 7B. The insert catheter 1300′ can incorporate any of the features of the insert catheter 1300 except the differences described with reference to FIG. 7B. For example, the insert catheter 1300′ can include a tubular body 1305′ including a distal end 1305a′, an inner liner 1320′, a reinforcing element 1340′, and an outer jacket 1360′. The exit port 1350′ can be in communication with the lumen 1390′. The exit port 1350′ can allow for the introduction and/or removal of a guidewire into and/or out of the lumen 1390′. This can beneficially allow for the advancement and/or retraction of the guidewire through the lumen 1390′ without blocking the proximal end of the insert catheter 1300′.

In some cases, the exit port 1350′ can be located on an inner (for example, concave) side of a pre-shaped tip of the insert catheter 1300′. The exit port 1350′ can be located on a same side as a concave side of a pre-shaped tip. In other implementations, the exit port 1350′ can be located elsewhere around a circumference of the catheter (e.g., on the convex side of a pre-shaped tip). Other example insert catheters, including other insert catheters with different pre-shaped tips, can incorporate any features of the insert catheter 1300′. In some cases, the exit port 1350′ can be located about 10 cm to about 100 cm, about 20 cm to about 90 cm, about 30 cm to about 80 cm, about 40 cm to about 70 cm, or about 50 cm to about 60 cm, from the distal end 1305a′ of the insert catheter 1300′. In some implementations, any of the insert catheter examples disclosed herein, for example, the insert catheter 1400, 1500, 1600, can incorporate the features of the insert catheter 1300′.

FIG. 8A shows an example of an insert catheter 1400. The insert catheter 1400 can include a distal portion 1400a, a transitional portion 1400b, and a distal portion 1400c. The insert catheter 1400 can include a tubular body 1405 having a proximal end 1405a and a distal end 1405b. The tubular body 1405 can also include an inner liner, a reinforcing element, an outer jacket, and/or a lumen extending between the proximal end 1405a and the distal end 1405b. The inner liner, reinforcing element, and/or outer jacket of the insert catheter 1400 can be similar or identical to the inner liner 1320, reinforcing element 1340, and/or outer jacket 1360 of the insert catheter 1300, which is described in relation to FIGS. 7A-7C. In some cases, the tubular body 1405 can have a length from about 120 cm to about 160 cm. For example, the tubular body 1405 can have a length from about 130 cm to about 150 cm, and/or from about 135 cm to about 145 cm, and in some cases about 137 cm, 139 cm, 140 cm, and/or 143 cm.

In some cases, the proximal end 1405a of the tubular body 1405 can be connected to another medical device. For example, the proximal end 1405a of the tubular body 1405 can be connected to a manifold or hub 1450. In some cases, a strain relief 1452 can be positioned between the proximal end 1405a of the tubular body 1405 and the hub 1450. The strain relief 1452 can beneficially prevent kinking of the tubular body 1405.

The insert catheter 1400 can also include a pre-shaped tip (or region) 1430. As shown in FIG. 8B, the pre-shaped tip 1430 can include more than one curve (or concave sides and/or at least one convex sides). For example, the pre-shaped tip 1430 can include a first curve defined by a first concave side 1432a and a first convex side 1423a. The first curve can be the proximal most curve. The pre-shaped tip 1430 can include a second curve defined by a second concave side 1432b and a second convex side 1434b. The second curve can be more distal than the first curve. The pre-shaped tip 1430 can include a third curved defined by a third concave side 1432c and a third convex side 1424c. The third curve can be a distal most curve. In some implementations, the insert catheter 1400 has the pre-shaped tip of a Simmons catheter.

The portion of the tubular body 1405 immediately on the proximal side of the first curve and the portion of the tubular body 1405 immediately on the distal side of the first curve (i.e., the proximal most portion of the tip 1430) can establish an angle A3. The angle A3 can be between about 120° and about 180°. For example, the angle A3 can be between about 130° and about 170°, between about 140° and about 160°, and/or between about 150° and about 160°. The portion of the tubular body 1405 on the proximal side of the first curve can define a longitudinal axis of the catheter 1400. The portion of the tubular body 1405 on the distal side of the first curve can be at an oblique takeoff angle A3 from the longitudinal axis of the catheter 1400.

The second curve results in the tubular body 1405 in the tip 1430 reversing back toward the longitudinal axis. The portions of the tubular body 1405 immediately on the proximal and distal sides of the second curve can establish an angle A2. The angle A2 can be between about 1° and about 50°. For example, the angle A2 can be between about 5° and about 40°, between about 10° and about 30°, and/or between about 15° and about 25°. The second curve can allow catheter advancement by pulling rather than pushing, leading to easier and faster catheter placement.

The third curve turns the tubular body 1405 in tip 1430 away from the first curve. The portions of the tubular body 1405 immediately on the proximal and distal sides of the third curve can establish an angle A1. The angle A1 can be between about 90° and about 140°. For example, the angle A2 can be between about 95° and about 135°, between about 100° and about 130°, and/or between about 110° and about 120°. The third curve can help in the anchorage at the aorta.

The pre-shaped tip 1430 can establish a length L2 between the longitudinal axis of the tubular body 1405 and the distal end 1405b. The length L2 can be the shortest distance measured from the longitudinal axis to the distal end 1405b. In some cases, the length L2 can be from about 1 cm. to about 6 cm. For example, the length L2 can be from about 1.5 cm. to about 5.5. cm., from about 2 cm. to about 5 cm., from about 2.5 cm. to about 4.5 cm., and/or from about 3 cm. to about 4 cm.

In some cases, the reinforcing element of the tubular body 1405 can extend an entire length of the tubular body 1405. In other cases, the reinforcing element can terminate proximal to the distal end 1405b of the tubular body 1405. For example, the reinforcing element can extend from the proximal end 1405a distally toward the distal end 1405b and terminate from about 6 cm to about 8.5 cm, from about 6.5 cm to about 7.5 cm, and/or from about 7 cm to about 7.5 cm, from the distal end 1405b. In some cases, the reinforcing element may terminate at around (e.g., +/−0.15 cm) an apex of one of the curves of the pre-shaped tip 1430, such as the apex of the second curve.

At least a portion of the insert catheter 1400 may taper. For example, the distal portion 1400a can taper in a distal direction. That is, the outer diameter of the insert catheter 1400 along the distal portion 1400a may decrease in a distal direction so that the outer diameter at the distal end 1405b is smaller than the outer diameter of more proximal portions of the insert catheter 1400. In some cases, the outer diameter of the insert catheter may taper from between about 0.08 in. and 0.085 in. to between about 0.060 in. and 0.065 in., over a length of between about 5 cm to about 7 cm. In some cases, an entire length of the intermediate portion 1400b can have an outer diameter of between about 0.08 in and 0.085 in. An entire length of the proximal portion 1400c can have an outer diameter of between about 0.075 in and 0.085 in.

As shown in FIG. 8C, the insert catheter 1400 may be positioned within the vasculature of a patient. For example, the insert catheter 1400 may be tracked up to and positioned within the aortic arch 1490. Once inside the aortic arch 1490, the position of the insert catheter 1400 can be adjusted so that the distal end 1405b of the insert catheter is positioned at the origin of or inside the right brachiocephalic artery 1482, the left common carotid artery (CCA) 1480, and/or the left subclavian artery 1484. For example, the distal end 1405b of the insert catheter 1400 can be positioned within the CCA 1480. A guidewire can be advanced though the lumen of the insert catheter 1400. The guidewire can extend beyond the distal end 1405b of the insert catheter 1400 into more distal portions of the vasculature.

As previously described herein, one or more catheters may be advanced over the insert catheter 1400 and/or a guidewire. The position of the insert catheter 1400 within the aortic arch 1490 can beneficially provide support for the catheters to be advanced into the arteries adjacent to the aortic arch. In some cases, a guide sheath or catheter 1472, which can be similar or identical to the guide sheath 1222, can be advanced over the insert catheter 1400. The guide catheter 1472 can be advanced beyond the distal end 1405b of the insert catheter 1400. This can beneficially allow the guide catheter 1472 to be positioned into more distal portions of the vasculature.

The torsional stiffness of the insert catheter 1400 can be optimized to allow the insert catheter 1400 to transmit torque effectively from the proximal end 1405a of the insert catheter 1400 to the distal end 1405b, enabling precise control during navigation. The torsional stiffness can be measured in terms of torque per rotation (e.g., N*cm/rotation) and is sufficient to allow the catheter to self-orient and navigate past challenging anatomical features. The insert catheter's ability to twist and align with the natural curvature of the vessels can reduce the need for manual rotation at the proximal end 1405a, improving procedural efficiency and reducing the risk of vessel injury. The combination of the torsional stiffness and shape of the pre-shaped tip (e.g., the curvature and/or angled distal face of the pre-shaped tip) can allow for the insert catheter to rotate and align with the natural curvature of the vessels to self-orient along a path of least resistance in the vasculature. This can allow for navigation through challenging anatomical structures with minimal or no additional torque applied by a user (e.g., to the proximal end 1405a of the insert catheter 1400).

In some cases, the insert catheter 1400 can have an average torque response from about 0.001 Ncm/deg to about 0.008 Ncm/deg. In some cases, the catheter 1400 can have an average torque response from about 0.002 Ncm/deg to about 0.006 Ncm/deg, from about 0.0025 Ncm/deg to about 0.0045 Ncm/deg, and/or from about 0.003 Ncm/deg to about 0.004 Ncm/deg. In some cases, the catheter 1400 can have an average torque response from about 0.0030 Ncm/deg to about 0.0034 Ncm/deg.

In some embodiments, the torsional stiffness over 1 rotation (e.g., rotating one end of the insert catheter 1400 by about 360 degrees and measuring the corresponding rotation at or near the other end of the insert catheter 1400) can be between about 0.90 Ncm/rotation and 1.5 Ncm/rotation. For example, the torsional stiffness of the insert catheter 1400 over 1 rotation can be between about 0.95 Ncm/rotation and 1.45 Ncm/rotation, between about 1.0 Ncm/rotation and 1.40 Ncm/rotation, between about 1.05 Ncm/rotation and 1.35 Ncm/rotation, between about 1.10 Ncm/rotation and 1.20 Ncm/rotation.

In some cases, the distal most 5 mm of the insert catheter 1400 can have an average flexibility between about 0.0005 mm/gF and about 0.0025 mm/gF (e.g., about 0.00190).

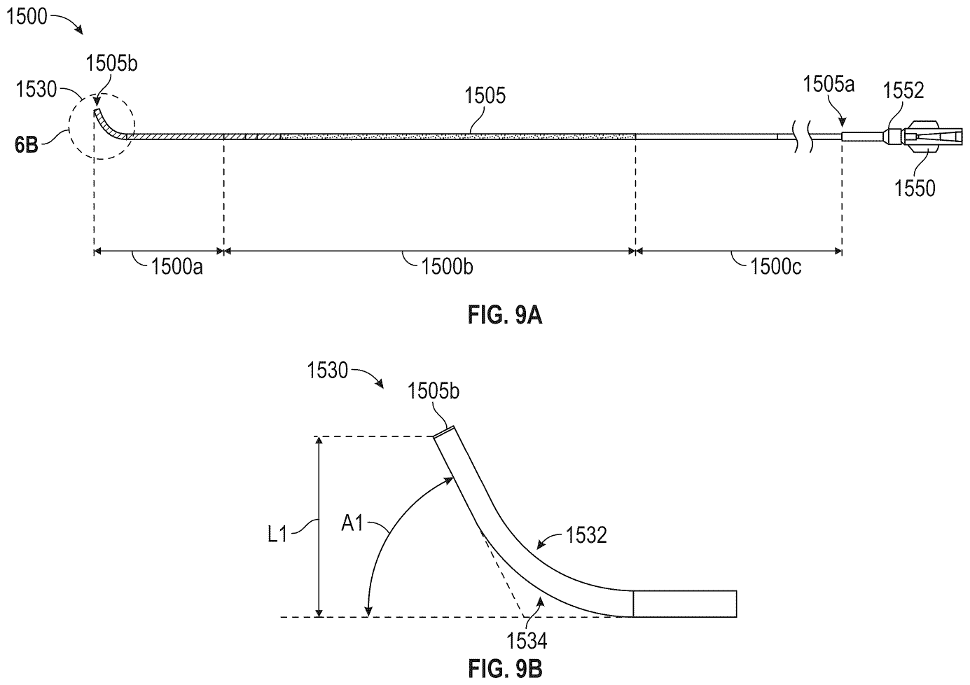

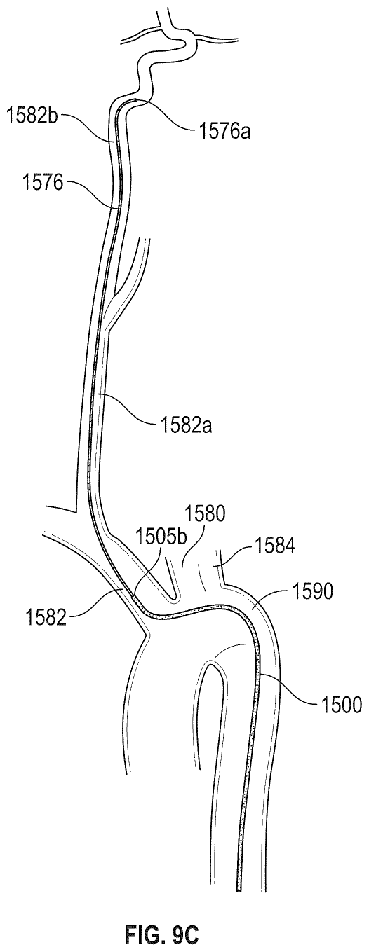

FIG. 9A shows an example of an insert catheter 1500. The insert catheter 1500 can include a distal portion 1500a, a transitional portion 1500b, and a distal portion 1500c. The insert catheter 1500 can include a tubular body 1505 having a proximal end 1505a and a distal end 1505b. The tubular body 1505 can include an inner liner, a reinforcing element, an outer jacket, and/or a lumen extending between the proximal end 1505a and the distal end 1505b. The inner liner, reinforcing element, and/or outer jacket of the insert catheter 1500 can be similar or identical to the inner liner 1320, reinforcing element 1340, and/or outer jacket 1360 of the insert catheter 1300, which is described in relation to FIGS. 7A-7C. In some implementations, the tubular body 1505 can have a length from about 120 cm to about 160 cm. For example, the tubular body 1405 can have a length from about 130 cm to about 150 cm, and/or from about 135 cm. to about 145 cm, and in some cases about 137 cm, 139 cm, 140 cm, and/or 143 cm.

In some implementations, the proximal end 1505a of the tubular body 1505 can be connected to another medical device. For example, the proximal end 1505a of the tubular body 1505 can be connected to a manifold or hub 1550. In some implementations, a strain relief 1552 can be positioned between the proximal end 1505a of the tubular body 1505 and the hub 1550. The strain relief 1552 can beneficially prevent kinking of the tubular body 1505.

The insert catheter 1500 can include a pre-shaped tip (or region) 1530. As shown in FIG. 9B, the pre-shaped tip 1530 can include at least one concave side and/or at least one convex side. For example, the pre-shaped tip 1530 can include a curve defined by a concave side 1532 and a convex side 1534. In some implementations, the insert catheter 1500 can have a pre-shaped tip of a vertebral (VERT or VRT) catheter.

The portion of the tubular body 1505 immediately on the proximal side of the curve and the portion of the tubular body 1505 immediately on the distal side of the curve (i.e., the proximal most portion of the tip 1530) can establish an angle A1. The angle A1 can be between about 30° and about 90°. For example, the angle A1 can be between about 40° and about 80°, between about 50° and about 70°, and/or between about 60° and about 65°. The portion of the tubular body 1505 on the proximal side of the curve can define a longitudinal axis of the catheter 1500. The portion of the tubular body 1505 on the distal side of the curve (in the tip 1530) can be at an oblique takeoff angle A1 from the longitudinal axis of the catheter 1530.

The pre-shaped tip 1530 can establish a length L1 between the longitudinal axis of the tubular body 1505 and the distal end 1505b. The length L1 can be the shortest distance measured from the longitudinal axis to the distal end 1505B. In some cases, the length L1 can be from about 0.1 cm to about 3 cm. For example, the length L1 can be from about 0.2 cm to about 2.5 cm, from about 0.3 cm to about 2 cm, from about 0.5 cm to about 1.5 cm, and/or from about 1 cm to about 1.3 cm.

In some cases, the reinforcing element of the tubular body 1505 can extend an entire length of the tubular body 1505. In other cases, the reinforcing element can terminate proximal to the distal end 1505b of the tubular body 1505. For example, the reinforcing element can extend from the proximal end 1505a distally toward the distal end 1505b and terminate from about 6 cm. to about 8.5 cm, from about 6.5 cm. to about 7.5 cm., and/or from about 7 cm to about 7.5 cm., from the distal end 1505b. In some cases, the reinforcing element may terminate at or around (e.g., +/−0.15 cm) an apex of the curve of the pre-shaped tip 1530.

At least a portion of the insert catheter 1500 may taper. For example, the distal portion 1500a can taper in a distal direction. That is, the outer diameter of the insert catheter 1500 along the distal portion 1500a may decrease in a distal direction so that the outer diameter at the distal end 1505b is smaller than the outer diameter of more proximal portions of the insert catheter 1500. In some cases, the outer diameter of the insert catheter may taper from between about 0.08 in and 0.085 in. to between about 0.060 in. and 0.065 in., over a length of between about 1 cm to about 3 cm. In some cases, an entire length of the intermediate portion 1500b can have an outer diameter of between about 0.08 in. and 0.085 in. An entire length of the proximal portion 1500c can have an outer diameter of between about 0.075 in. and 0.085 in.

As shown in FIG. 9C, the insert catheter 1500 may be positioned within the vasculature of a patient. For example, the insert catheter 1500 may be tracked up to and positioned within the aortic arch 1590. Once inside the aortic arch 1590, the position of the insert catheter 1500 can be adjusted so that the distal end 1505b of the insert catheter 1500 is positioned at the origin of or inside the right brachiocephalic artery 1582, the left common carotid artery (CCA) 1580, and/or the left subclavian artery 1584. For example, the distal end 1505b of the insert catheter 1500 can be positioned within the right brachiocephalic artery 1582.

A guidewire 1576 can be advanced though the lumen of the insert catheter 1500. The guidewire 1576 can extend beyond the distal end 1505b of the insert catheter 1500 into more distal portions of the vasculature. For example, a distal end 1576a of the guidewire 1576 can be positioned at least as far as the right middle cerebral artery (RMCA) which branches from the right internal carotid artery (RICA) 1582b, which in turn branches from the right common carotid artery (RCCA) 1582a.

As previously described herein, one or more catheters may be advanced over the insert catheter 1500 and/or the guidewire 1576. The position of the insert catheter 1500 within the aortic arch 1590 can beneficially provide support for the catheters to be advanced into the arteries adjacent to the aortic arch 1590. In some cases, a guide sheath or catheter, which can be similar or identical to the guide sheath 1222, can be advanced over the insert catheter 1500 and/or the guidewire 1576. The guide catheter can be advanced beyond the distal end 1505b of the insert catheter 1500. This can beneficially allow the guide catheter to be positioned into more distal portions of the vasculature.

The torsional stiffness of the insert catheter 1500 can be optimized to allow the insert catheter 1500 to transmit torque effectively from the proximal end 1505a of the insert catheter 1500 to the distal end 1505b, enabling precise control during navigation. The torsional stiffness can be measured in terms of torque per rotation (e.g., N*cm/rotation) and is sufficient to allow the catheter to self-orient and navigate past challenging anatomical features. The insert catheter's ability to twist and align with the natural curvature of the vessels can reduce the need for manual rotation at the proximal end 1505a, improving procedural efficiency and reducing the risk of vessel injury. The combination of the torsional stiffness and shape of the pre-shaped tip (e.g., the curvature and/or angled distal face of the pre-shaped tip) can allow for the insert catheter to rotate and align with the natural curvature of the vessels to self-orient along a path of least resistance in the vasculature. This can allow for navigation through challenging anatomical structures with minimal or no additional torque applied by a user (e.g., to the proximal end 1505a of the insert catheter 1500).

In some cases, the insert catheter 1500 can have an average torque response from about 0.0005 Ncm/deg to about 0.003 Ncm/deg. In some cases, the catheter 1500 can have an average torque response from about 0.00055 Ncm/deg to about 0.0025 Ncm/deg, from about 0.001 Ncm/deg to about 0.0020 Ncm/deg, and/or from about 0.0011 Ncm/deg to about 0.0015 Ncm/deg.

In some embodiments, the torsional stiffness over 1 rotation (e.g., one end of the insert catheter 1500 by about 360 degrees and measuring the corresponding rotation at or near the other end of the insert catheter 1500) can be between about 0.10 Ncm/rotation and 1.0 Ncm/rotation. For example, the torsional stiffness of the insert catheter 1500 over 1 rotation can be between about 0.20 Ncm/rotation and 0.80 Ncm/rotation, between about 0.30 Ncm/rotation and 0.60 Ncm/rotation, between about 0.35 Ncm/rotation and 0.50 Ncm/rotation, and/or between about 0.40 Ncm/rotation and 0.45 Ncm/rotation.

In some cases, the distal most 5 mm of the insert catheter 1500 can have an average flexibility between about 0.0005 mm/gF and about 0.0025 mm/gF (e.g., about 0.00190).

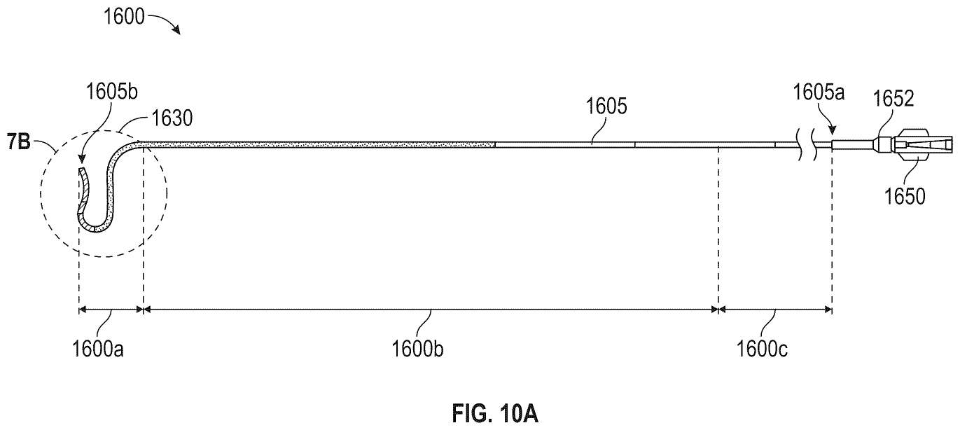

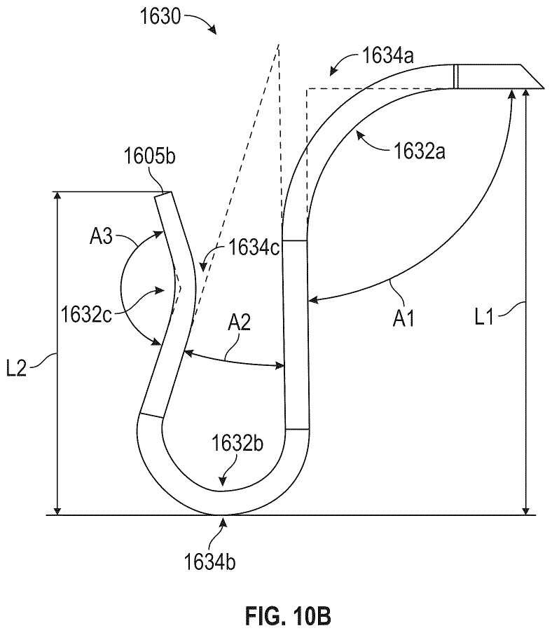

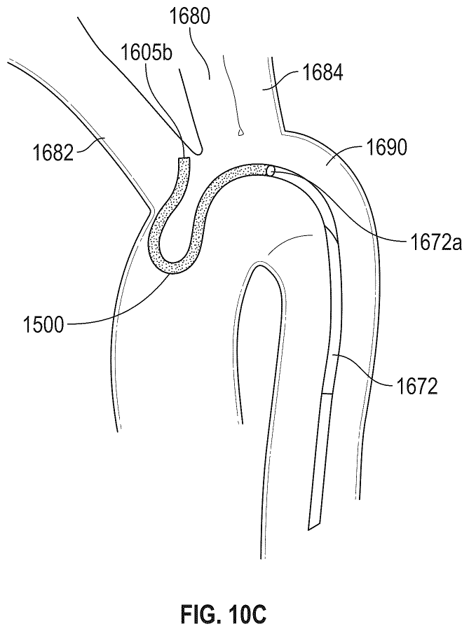

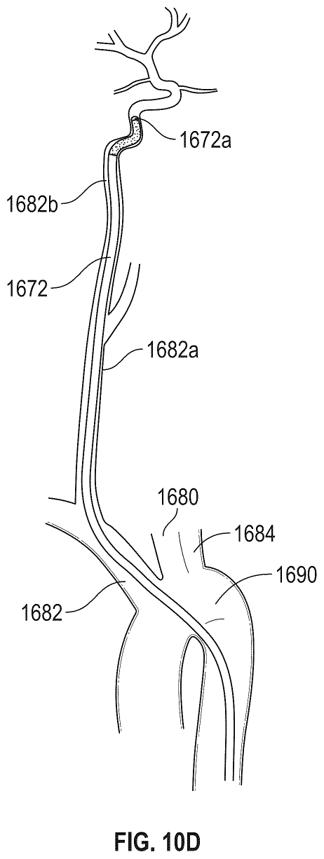

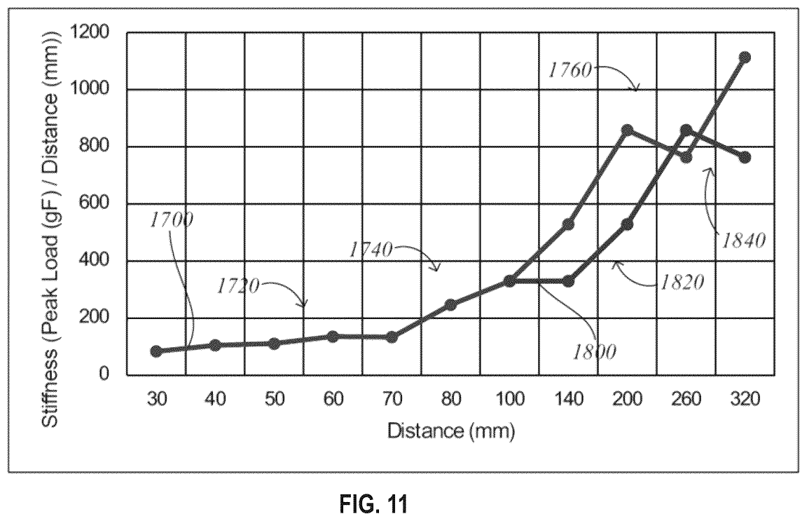

FIG. 10A shows an example of an insert catheter 1600. The insert catheter 1600 can include a distal portion 1600a, a transitional portion 1600b, and a distal portion 1600c. The insert catheter 1600 can include a tubular body 1605 having a proximal end 1605a and a distal end 1605b. The tubular body 1605 can also include an inner liner, a reinforcing element, an outer jacket, and/or a lumen extending between the proximal end 1605a and the distal end 1605b. The inner liner, reinforcing element, and/or outer jacket of the insert catheter 1600 can be similar or identical to the inner liner 1320, reinforcing element 1340, and/or outer jacket 1360 of the insert catheter 1300, which is described in relation to FIGS. 7A-7C. In some cases, the tubular body 1605 can have a length from about 120 cm to about 160 cm. For example, the tubular body 1605 can have a length from about 130 cm to about 150 cm, and/or from about 135 cm to about 145 cm, and in some cases about 137 cm, 139 cm, 140 cm, and/or 143 cm.

In some cases, the proximal end 1605a of the tubular body 1605 can be connected to another medical device. For example, the proximal end 1605a of the tubular body 1605 can be connected to a manifold or hub 1650. In some cases, a strain relief 1652 can be positioned between the proximal end 1605a of the tubular body 1605 and the hub 1650. The strain relief 1652 can beneficially prevent kinking of the tubular body 1605.

The insert catheter 1600 can also include a pre-shaped tip (or region) 1630. As shown in FIG. 10B, the pre-shaped tip 1630 can include at least one concave side and/or at least one convex side. For example, the pre-shaped tip 1630 can include a first curve defined by a first concave side 1632a and a first convex side 1634a, a second curve defined by a second concave side 1632b and a second convex side 1634b, and a third curved defined by a third concave side 1632c and a third convex side 1634c. In some implementations, the insert catheter 1600 can have a pre-shaped tip of a Vitek (VTK) catheter.

The portion of the tubular body 1605 immediately on the proximal side of the first curve and the portion of the tubular body 1505 immediately on the distal side of the first curve (i.e., the proximal most portion of the tip 1630) can establish an angle A1. The angle A1 can be between about 40° and about 140°. For example, the angle A1 can be between about 50° and about 130°, between about 60° and about 120°, between about 70° and about 110°, between about 80° and about 100°, and/or between about 85° and about 95°. The portion of the tubular body 1605 on the proximal side of the first curve can define a longitudinal axis of the catheter 1600. The portion of the tubular body 1605 on the distal side of the first curve (in the tip 1530) can be at a takeoff angle A1 from the longitudinal axis of the catheter 1600.

The second curve results in the tubular body 1605 in the tip 1630 reversing back (or U-turning) toward the longitudinal axis and converging toward the portion of the tip 1630 between the first curve and the second curve. The portions of the tubular body 1605 immediately on the proximal and distal sides of the second curve can establish an angle A2. The angle A2 can be between about 1° and about 70°. For example, the angle A2 can be between about 5° and about 60°, between about 10° and about 40°, and/or between about 15° and about 20°.

The third curve again turns the tubular body 1605 in the distal most portion of the tip 1630 away from the portion of the tubular body 1605 between the first curve and the second curve so that the distal end 1605b points distally. The portions of the tubular body 1605 immediately on the proximal and distal sides of the third curve can establish an angle A3. The angle A3 can be between about 100° and about 200°. For example, the angle A1 can be between about 110° and about 190°, between about 120° and about 180°, between about 130° and about 170°, and/or between about 140° and about 150°.

The pre-shaped tip 1630 can establish a length L1 between the longitudinal axis of the tubular body 1605 and the second convex side 1634b. The length L1 can be the shortest distance measured from the longitudinal axis to a tangent to the curvature of the second convex side 1634b that is parallel to the longitudinal axis. In some cases, the length L1 can be from about 1 cm. to about 6 cm. For example, the length L1 can be from about 1.5 cm. to about 5.5. cm., from about 2 cm. to about 5 cm., from about 2.5 cm. to about 4.5 cm., and/or from about 3 cm. to about 4 cm.

The pre-shaped tip 1630 can establish a length L2, the shortest distance between the distal end 1605b of the tubular body 1605 and the tangent to the curvature of the second convex side 1634b that is parallel to the longitudinal axis. In some cases, the length L2 can be from about 1 cm. to about 5 cm. For example, the length L1 can be from about 1.5 cm. to about 4.5. cm., from about 2 cm. to about 5 cm., from about 2.3 cm. to about 4.7 cm., and/or from about 2.5 cm. to about 4.5 cm.

In some cases, the reinforcing element of the tubular body 1605 can extend an entire length of the tubular body 1605. In other cases, the reinforcing element can terminate proximal to the distal end 1605b of the tubular body 1605. For example, the reinforcing element can extend from the proximal end 1605a distally toward the distal end 1605b and terminate from about 1 cm. to about 4 cm, from about 1.2 cm. to about 3.8 cm., from about 1.5 cm to about 3.5 cm., and/or from about 1.8 cm. to about 3.2 cm., from the distal end 1605b. In some cases, the reinforcing element may terminate at or around (e.g., +/−0.15 cm) an apex of one of the concave and/or convex sides, such as the second curve of the pre-shaped tip 1630.

At least a portion of the insert catheter 1600 may taper. For example, the distal portion 1600a can taper in a distal direction. That is, the outer diameter of the insert catheter 1600 along the distal portion 1600a may decrease in a distal direction so that the outer diameter at the distal end 1605b is smaller than the outer diameter of more proximal portions of the insert catheter 1600. In some cases, the outer diameter of the insert catheter may taper from between about 0.08 in and 0.085 in. to between about 0.060 in. and 0.065 in., over a length of between about 1 cm to about 4 cm. In some cases, an entire length of the intermediate portion 1600b can have an outer diameter of between about 0.08 in and 0.085 in. An entire length of the proximal portion 1600c can have an outer diameter of between about 0.075 in and 0.085 in.

As shown in FIG. 10C, the insert catheter 1600 may be positioned within the vasculature of a patient. For example, the insert catheter 1600 may be tracked up to and positioned within the aortic arch 1690. Once inside the aortic arch 1690, the position of the insert catheter 1600 can be adjusted so that the distal end 1605b of the insert catheter 1600 is positioned at the origin of or inside the right brachiocephalic artery 1682, the left common carotid artery (CCA) 1680, and/or the left subclavian artery 1684. For example, the distal end 1605b of the insert catheter 1600 can be positioned within the right brachiocephalic artery 1682.

A guidewire can be advanced though the lumen of the insert catheter 1600. The guidewire can extend beyond the distal end 1605b of the insert catheter 1600 into more distal portions of the vasculature. For example, a distal end of the guidewire can be positioned at least as far as the right middle cerebral artery (RMCA) which branches from the right internal carotid artery (RICA) 1682b, which in turn branches from the right common carotid artery (RCCA) 1682a, which are shown in FIG. 10D.

As previously described herein, one or more catheters may be advanced over the insert catheter 1600 and/or the guidewire. The position of the insert catheter 1600 within the aortic arch 1690 can beneficially provide support for the catheters to be advanced into the arteries adjacent to the aortic arch 1690. In some cases, a guide sheath or catheter 1672, which can be similar or identical to the guide sheath 1222, can be advanced over the insert catheter 1600 and/or the guidewire. The guide catheter 1672 can be advanced beyond the distal end 1605b of the insert catheter 1600. For example, a distal end 1672a of the guide catheter 1672 can be positioned at least as far as the right middle cerebral artery (RMCA) which branches from the right internal carotid artery (RICA) 1682b, which in turn branches from the right common carotid artery (RCCA) 1682a,

The torsional stiffness of the insert catheter 1600 can be optimized to allow the insert catheter 1600 to transmit torque effectively from the proximal end 1605a of the insert catheter 1600 to the distal end 1605b, enabling precise control during navigation. The torsional stiffness can be measured in terms of torque per rotation (e.g., N*cm/rotation) and is sufficient to allow the catheter to self-orient and navigate past challenging anatomical features. The insert catheter's ability to twist and align with the natural curvature of the vessels can reduce the need for manual rotation at the proximal end 1605a, improving procedural efficiency and reducing the risk of vessel injury. The combination of the torsional stiffness and shape of the pre-shaped tip (e.g., the curvature and/or angled distal face of the pre-shaped tip) can allow for the insert catheter to rotate and align with the natural curvature of the vessels to self-orient along a path of least resistance in the vasculature. This can allow for navigation through challenging anatomical structures with minimal or no additional torque applied by a user (e.g., to the proximal end 1605a of the insert catheter 1600).

In some cases, the insert catheter 1600 can have an average torque response from about 0.001 Ncm/deg to about 0.008 Ncm/deg. In some cases, the catheter 1600 can have an average torque response from about 0.002 Ncm/deg to about 0.006 Ncm/deg, from about 0.0025 Ncm/deg to about 0.005 Ncm/deg, and/or from about 0.003 Ncm/deg to about 0.004 Ncm/deg. In some cases, the catheter 1400 can have an average torque response from about 0.0030 Ncm/deg to about 0.0034 Ncm/deg.

In some embodiments, the torsional stiffness over 1 rotation (e.g., one end of the insert catheter 1600 by about 360 degrees and measuring the corresponding rotation at or near the other end of the insert catheter 1600) can be between about 0.5 Ncm/rotation and 1.5 Ncm/rotation. For example, the torsional stiffness of the insert catheter 1400 over 1 rotation can be between about 0.80 Ncm/rotation and 1.45 Ncm/rotation, between about 0.90 Ncm/rotation and 1.40 Ncm/rotation, between about 0.95 Ncm/rotation and 1.35 Ncm/rotation, between about 1.0 Ncm/rotation and 1.15 Ncm/rotation.

In some cases, the distal most 5 mm of the insert catheter 1600 can have an average flexibility between about 0.0005 mm/gF and about 0.0025 mm/gF (e.g., about 0.00156).

### Flexibility Profile

FIG. 11 illustrates example flexibility profiles of a distal portion of various insert catheter examples, such as insert catheters 1300, 1400, 1500, and/or 1600, from a distal location of the insert catheter to more proximal locations of the insert catheter. The x-axis represents the length of the catheter from the distal end (0 mm) toward the proximal end (that is, toward the right hand side of the graph). In some cases, the flexibility profile of the insert catheter can be determined using a cantilever beam test. The cantilever beam test can be conducted along the length of the insert catheter to characterize the stiffness and/or rate of change of stiffness along different axial points and/or portions of the insert catheter. The stiffness and/or rate of change of stiffness along different axial points and/or portions of the insert catheter can be related to the anatomical area that the catheter is designed to navigating through.

In the present application, the flexibility of the insert catheters 1400, 1500, and 1600, was tested at various points along the length of the tubular bodies 1405, 1505, and 1605 respectively. According to one implementation of the cantilever beam test, the insert catheter can be secured at a 5 mm distance from the target location to be measured. This is the gage length (e.g., 5 mm) used by the cantilever beam test. A force (e.g., peak load) is then applied to cause a 4 mm displacement at the target location of the insert catheter. The amount of force applied can measured at each sampling location (e.g., every 5 mm, 10 mm, 15 mm, 20 mm, 25 mm, 30 mm, 40 mm, 50 mm, 60 mm, etc.) to create a flexibility profile for the insert catheter 1500.

The flexibility profiles 1700 and 1800 shown in FIG. 11 illustrate the stiffness (Gf/mm) experienced at various locations along the length of the tubular bodies 1405, 1505, and 1605, that are proximal to the pre-shaped tips 1430, 1530, 1630, as described with reference to FIGS. 8A-10D. The flexibility profiles 1700 and/or 1800 represent the peak load (gF) along the length of the tubular bodies 1405, 1505, 1605, measured by determining the amount of force required to displace the target location by 4 mm, wherein the peak load is measured within the 4 mm displacement. The flexibility of the insert catheters 1400, 1500, 1600, may be the same such that the flexibility profile 1700 may correspond to the flexibility profile of the insert catheters 1400, 1500, 1600. In some cases, however, the insert catheters 1400, 1500, 1600 may have different flexibility profiles. For example, the flexibility profile 1800 may correspond to the flexibility profile of the insert catheter 1400 and/or the insert catheter 1600.

As an example, the flexibility profile 1700 may correspond to the flexibility profile of the insert catheter 1500. In some cases, the average stiffness of the insert catheters (measured in peak load/distance) along an area where the braid is positioned can be from about 300 gF/mm to about 700 gF/mm. For example, the average stiffness can be from 350 gF/mm to about 650 gF/mm, from about 400 gF/mm to about 600 gF/mm, and/or from about 450 gF/mm to about 500 gF/mm. The flexibility profile 1700 can include a first section 1720, a second section 1740, and a third section 1760. The first section 1720 illustrates a peak load experienced by a distal portion 1500a of the insert catheter 1500 (excluding the pre-shaped tip 1530). For example, the first section 1720 can correspond to the distal-most 6 cm to 10 cm of the insert catheter 1500, and in some cases 7 cm to 9 cm, and/or 7.5 cm to 8.5 cm. The distal portion 1500a of the insert catheter 1500 can have a stiffness (measured in peak load value/distance) between about 1 gF/mm to about 250 gF, between about 1 gF/mm to about 200 gF, between about 10 gF/mm to about 200 gF/mm, between about 50 gF/mm to about 180 gF/mm, between about 100 gF/mm to about 150 gF/mm, and any value in between the ranges listed including endpoints. In some cases, and as shown by section 1720, the stiffness of the distal portion 1500a of the insert catheter 1500 can increase from or about 80 gF/mm to about 140 gF/mm over a length of about 4 cm.

The second section 1740 illustrates a peak load experienced by a transitional portion 1500b of the insert catheter 1500 as described with reference to FIGS. 9A and 9B. The transitional portion 1500b of the insert catheter 1500 can include a length from about 10 cm to about 20 cm. For example, the transitional portion 1500b of the insert catheter 1500 can have a length from about 12 cm to about 18 cm, from about 14 cm to about 16 cm, and/or from about 14.5 cm to about 15.5 cm. The transitional portion 1500b of the insert catheter 1500 can have a stiffness (measured in peak load/distance) value between about 150 gF/mm to about 900 gF/mm, between about 300 gF/mm to about 800 gF/mm, between about 400 gF/mm to about 700 gF/mm, and any value in between the ranges listed including endpoints. In some cases, and as shown by section 1740, the stiffness of the transitional section 1500b of the insert catheter 1500 can increase from or about 150 gF/mm to about 900 gF/mm over a length of about 13 cm.

The third section 1760 illustrates a peak load experienced by a proximal portion 1500c of the insert catheter 1500 as described with reference to FIGS. 9A and 9B. The proximal portion 1500c of the insert catheter 1500 can include a length from about 100 cm to about 125 cm. For example, the proximal portion 1500c of the insert catheter 1500 can have a length from about 105 cm to about 120 cm, and/or from about 110 cm to about 115 cm. The proximal portion 1500c of the insert catheter 1500 can have a stiffness (measured in peak load value/distance) between about 750 gF/mm to about 1150 gF/mm, between about 800 gF/mm to about 1100 gF/mm, between about 900 gF/mm to about 1000 gF/mm, and any value in between the ranges listed including endpoints.

As previously described herein, the insert catheters 1400, 1500, 1600, may have different flexibility profiles. For example, the insert catheter 1400 and/or the insert catheter 1600, which have a longer pre-shaped tip than the insert catheter 1500, may have a flexibility profile 1800 different than the flexibility profile 1700. In such cases, at least a portion of the flexibility profile 1700 may correspond to the flexibility profile of the insert catheter 1400 and/or the insert catheter 1500. For example, as shown in FIG. 11, the flexibility profile 1700 may correspond to the flexibility profile of the insert catheters along a portion of the catheter extending from about 30 mm to about 100 mm of the distal end of the insert catheters. The flexibility profile 1800 may correspond to the flexibility profile of the insert catheters along a portion of the catheter extending from about 100 mm to about 320 mm of the distal end of the insert catheters.

The flexibility profile 1800 can include a first section 1820 and a second section 1840. The first section 1820 illustrates a peak load experienced by a transitional portion (e.g., 1400b, 1500b, 1600b) of the insert catheters as described with reference to FIGS. 9A-10D. The transitional portion of the insert catheters can include a length from about 10 cm to about 30 cm. For example, the transitional portions of the insert catheters can have a length from about 12 cm to about 28 cm, from about 14 cm to about 26 cm, and/or from about 14.5 cm to about 25.5 cm. A stiffness (measured in peak load value/distance) along the first section 1820 can be between about 250 gF/mm to about 1000 gF/mm, between about 300 gF/mm to about 800 gF/mm, between about 400 gF/mm to about 700 gF/mm, and any value in between the ranges listed including endpoints. In some cases, and as shown by the first section 1820, the stiffness of the transitional section of the insert catheters can increase from or about 300 gF/mm to about 900 gF/mm over a length of about 16 cm.

The second section 1840 illustrates a peak load experienced by a proximal portion (e.g., 1400c, 1500c, 1600c) of the insert catheters as described with reference to FIGS. 9A-10D. The proximal portion of the insert catheters can include a length from about 100 cm to about 125 cm. For example, the proximal portion of the insert catheters can have a length from about 105 cm to about 120 cm, and/or from about 110 cm to about 115 cm. The proximal portion of the insert catheters along the second section 1840 can have a stiffness (measured in peak load value/distance) between about 700 gF/mm to about 900 gF/mm, between about 750 gF/mm to about 850 gF/mm, between about 775 gF/mm to about 825 gF/mm, and any value in between the ranges listed including endpoints. In some cases, and as shown by the second section 1840, the stiffness of the proximal section of the insert catheters can decrease from or about 860 gF/mm to about 760 gF/mm over a length of about 6 cm.

### Additional Embodiments

In some embodiments, access for the catheter can be achieved using conventional techniques through an incision on a peripheral artery, such as right femoral artery, left femoral artery, right radial artery, left radial artery, right brachial artery, left brachial artery, right axillary artery, left axillary artery, right subclavian artery, or left subclavian artery. An incision can also be made on right carotid artery or left carotid artery in emergency situations.

Avoiding a tight fit between the guidewire 40 and inside diameter of guidewire lumen 28 enhances the slidability of the catheter over the guidewire. In ultra small diameter catheter designs, it may be desirable to coat the outside surface of the guidewire 40 and/or the inside surface of the wall defining lumen 38 with a lubricous coating to minimize friction as the catheter 10 is axially moved with respect to the guidewire 40. A variety of coatings may be utilized, such as Paralene, Teflon, silicone, polyimide-polytetrafluoroethylene composite materials or others known in the art and suitable depending upon the material of the guidewire or inner tubular wall 38.

In some embodiments, aspiration catheters which are adapted for intracranial applications generally have a total length in the range of from 60 cm to 250 cm, usually from about 135 cm to about 175 cm. The length of the proximal segment 33 will typically be from 20 cm to 220 cm, more typically from 100 cm to about 120 cm. The length of the distal segment 34 will typically be in the range from 10 cm to about 60 cm, usually from about 25 cm to about 40 cm.

In some examples, the catheters may be composed of any of a variety of biologically compatible polymeric resins having suitable characteristics when formed into the tubular catheter body segments. Exemplary materials include polyvinyl chloride, polyethers, polyamides, polyethylenes, polyurethanes, copolymers thereof, and the like. In one embodiment, both the proximal body segment 33 and distal body segment 34 will comprise a polyvinyl chloride (PVC), with the proximal body segment being formed from a relatively rigid PVC and the distal body segment being formed from a relatively flexible, supple PVC. Optionally, the proximal body segment may be reinforced with a metal or polymeric braid or other conventional reinforcing layer.

The proximal body segment will exhibit sufficient column strength to permit axial positioning of the catheter through a catheter having an inner diameter larger than an outer diameter of the proximal body segment 33 with at least a portion of the proximal body segment 33 extending beyond the larger catheter and into the patient's vasculature. The proximal body segment may have a shore hardness in the range from 50 D to 100 D, often being about 70 D to 80 D. Usually, the proximal shaft will have a flexural modulus from 20,000 psi to 1,000,000 psi, preferably from 100,000 psi to 600,000 psi. The distal body segment 34 will be sufficiently flexible and supple so that it may navigate the patient's more narrow distal vasculature. In highly flexible embodiments, the shore hardness of the distal body segment 34 may be in the range of from about 20 A to about 100 A, and the flexural modulus for the distal segment 34 may be from about 50 psi to about 15,000 psi.

The catheter body may further comprise other components, such as radiopaque fillers; colorants; reinforcing materials; reinforcement layers, such as braids or helical reinforcement elements; or the like. In particular, the proximal body segment may be reinforced in order to enhance its column strength and torqueability (torque transmission) while preferably limiting its wall thickness and outside diameter.

Usually, radiopaque markers will be provided at least at the distal end 14 and the transition region 32 at the distal end of proximal segment 33. One radiopaque marker comprises a metal band which is fully recessed within or carried on the outside of the distal end of the proximal body segment 33. Suitable marker bands can be produced from a variety of materials, including platinum, gold, and tungsten/rhenium alloy. Preferably, the radiopaque marker band will be recessed in an annular channel to produce a smooth exterior surface.

The proximal section 33 of tubular body 16 may be produced in accordance with any of a variety of known techniques for manufacturing interventional catheter bodies, such as by extrusion of appropriate biocompatible polymeric materials. Alternatively, at least a proximal portion or all of the length of tubular body 16 may comprise a polymeric or metal spring coil, solid walled hypodermic needle tubing, or braided reinforced wall, as is known in the microcatheter arts.

In a catheter intended for neurovascular applications, the proximal section 33 of body 16 will typically have an outside diameter within the range of from about 0.117 inches to about 0.078 inches. In one implementation, proximal section 33 has an OD of about 0.104 inches and an ID of about 0.088 inches. The distal section 34 has an OD of about 0.085 inches and an ID of about 0.070 inches.

Diameters outside of the preferred ranges may also be used, provided that the functional consequences of the diameter are acceptable for the intended purpose of the catheter. For example, the lower limit of the diameter for any portion of tubular body 16 in a given application will be a function of the number of fluid or other functional lumen contained in the catheter, together with the acceptable minimum aspiration flow rate and collapse resistance.

Tubular body 16 must have sufficient structural integrity (e.g., column strength or “pushability”) to permit the catheter to be advanced to distal locations without buckling or undesirable bending of the tubular body. The ability of the body 16 to transmit torque may also be desirable, such as to avoid kinking upon rotation, to assist in steering. The tubular body 16, and particularly the distal section 34, may be provided with any of a variety of torque and/or column strength enhancing structures. For example, axially extending stiffening wires, spiral wrapped support layers, braided or woven reinforcement filaments may be built into or layered on the tubular body 16. See, for example, U.S. Pat. No. 5,891,114 to Chien, et al., the disclosure of which is incorporated in its entirety herein by reference.

In many applications, the proximal section 33 will not be required to traverse particularly low profile or tortuous arteries. For example, the proximal section 33 will be mostly or entirely within the relatively large diameter catheter. The transition 32 can be located on the catheter shaft 16 to correspond approximately with or beyond the distal end of the large diameter catheter.

For certain applications, such as intracranial catheterizations, the distal section 34 is preferably at least about 5 cm or 10 cm long and small enough in diameter to pass through vessels as low as 3 mm or 2 mm or lower. The distal section may have a length of at least about 20 cm or 30 cm or 40 cm or more, depending upon the intended target vessel or treatment site.

The distal section, whether carried within the proximal section as an integrated device, or is a separate device to be inserted into the proximal section during a procedure, is substantially shorter than the proximal section. When the distal end of the distal section and the distal end of the proximal section are axially aligned, the proximal end of the distal section is spaced distally apart from the proximal end of the proximal section. The control element such as a control wire or tube spans the distance between the proximal end of the distal section and the proximal manifold or proximal control.

In the foregoing configuration, the proximal end of the distal section will generally be spaced apart distally from the proximal end of the proximal section by at least about 25%, and in some embodiments at least about 50% or 70% or more of the length of the proximal section.

### Disclaimer

Although the present invention has been described in terms of certain preferred embodiments, it may be incorporated into other embodiments by persons of skill in the art in view of the disclosure herein. The scope of the invention is therefore not intended to be limited by the specific embodiments disclosed herein, but is intended to be defined by the full scope of the following claims.

Accordingly, it is to be understood that the embodiments of the invention herein described are merely illustrative of the application of the principles of the invention. Reference herein to details of the illustrated embodiments is not intended to limit the scope of the claims, which themselves recite those features regarded as essential to the invention. The drawings are for the purpose of illustrating embodiments of the invention only, and not for the purpose of limiting it.

It is contemplated that various combinations or subcombinations of the specific features and aspects of the embodiments disclosed above may be made and still fall within one or more of the inventions. Further, the disclosure herein of any particular feature, aspect, method, property, characteristic, quality, attribute, element, or the like in connection with an embodiment can be used in all other embodiments set forth herein. Accordingly, it should be understood that various features and aspects of the disclosed embodiments can be combined with or substituted for one another in order to form varying modes of the disclosed inventions. Thus, it is intended that the scope of the present inventions herein disclosed should not be limited by the particular disclosed embodiments described above. Moreover, while the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents, and alternatives falling within the spirit and scope of the various embodiments described and the appended claims. Any methods disclosed herein need not be performed in the order recited. The methods disclosed herein include certain actions taken by a practitioner; however, they Can also include any third-party instruction of those actions, either expressly or by implication. For example, actions such as “deploying an instrument sterilized using the systems herein” include “instructing the deployment of an instrument sterilized using the systems herein.” In addition, where features or aspects of the disclosure are described in terms of Markush groups, those skilled in the art will recognize that the disclosure is also thereby described in terms of any individual member or subgroup of members of the Markush group.

The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers. For example, “about 10 nanometers” includes “10 nanometers.”

Any titles or subheadings used herein are for organization purposes and should not be used to limit the scope of embodiments disclosed herein.

The terms “approximately”, “about”, and “substantially” as used herein represent an amount or characteristic close to the stated amount or characteristic that still performs a desired function or achieves a desired result. For example, the terms “approximately”, “about”, and “substantially” may refer to an amount in certain embodiments that is within less than plus or minus 10% of, within less than plus or minus 5% of, within less than plus or minus 1% of, within less than plus or minus 0.1% of, and within less than plus or minus 0.01% of the stated amount or characteristic.

What is claimed is:

1. A neurovascular catheter for delivery to an ostium of the aortic arch, the neurovascular catheter comprising:
an elongate flexible body comprising a length of at least about 130 cm, the elongate flexible body comprising:
a distal portion comprising a pre-shaped tip,
a transitional portion proximal to the distal portion, and
a proximal portion proximal to the transitional;
wherein the elongate flexible body has an outer diameter of about 6 F.

2. The neurovascular catheter of claim 1, wherein the pre-shaped tip comprises:
a first curve defining a first concave side and a first convex side;
a second curve defining a second concave side and a second convex side; and
a third curve defining a third concave side and a third convex side;
wherein a longitudinal axis of the elongate flexible body and a first section of the pre-shaped tip distal to the first curve form an angle from about 120° to about 180°;
wherein the first section of the pre-shaped tip and a second section of the pre-shaped tip distal to the second curve form an angle from about 10° to about 50°; and
wherein the second section of the pre-shaped tip and a third section of the pre-shaped tip distal to the third curve form an angle from about 90° to about 140°.

3. The neurovascular catheter of claim 1, wherein a distance between a distal end of the elongate flexible body and a longitudinal axis of the elongate flexible body along an axis perpendicular to the longitudinal axis is between about 1 cm and about 6 cm.

4. The neurovascular catheter of claim 1, wherein the pre-shaped tip comprises:
a curve defining a concave side and a convex side;
wherein a longitudinal axis of the elongate flexible body and a section of the pre-shaped tip distal to the curve form an angle from about 30° to about 90°.

5. The neurovascular catheter of claim 1, wherein a distance between a distal end of the elongate flexible body and a longitudinal axis of the elongate flexible body along an axis perpendicular to the longitudinal axis is between about 0.1 cm and about 3 cm.

6. The neurovascular catheter of claim 1, wherein the pre-shaped tip comprises a length from about 16 cm to about 20 cm.

7. The neurovascular catheter of claim 1, wherein the pre-shaped tip comprises:
a first curve defining a first concave side and a first convex side;
a second curve defining a second concave side and a second convex side; and
a third curve defining a third concave side and a third convex side;
wherein a longitudinal axis of the elongate flexible body and a first section of the pre-shaped tip distal to the first curve form an angle from about 40° to about 140°;
wherein the first section of the pre-shaped tip and a second section of the pre-shaped tip distal to the second curve form an angle from about 10° to about 70°; and
wherein the second section of the pre-shaped tip and a third section of the pre-shaped tip distal to the third curve form an angle from about 100° to about 200°.

8. The neurovascular catheter of claim 1, wherein a distance between a longitudinal axis of the elongate flexible body and an apex of the second convex side along an axis perpendicular to the longitudinal axis is between about 1 cm and about 6 cm.

9. The neurovascular catheter of claim 1, wherein a distance between a distal tip of the elongate flexible body and an apex of the second convex side along an axis perpendicular to the longitudinal axis is between about 1 cm and about 5 cm.

10. The neurovascular catheter of claim 1, wherein the ostium of the aortic arch branches into of the right brachiocephalic artery, the left common carotid artery, and the left subclavian artery.

11. The neurovascular catheter of claim 1, wherein a stiffness of the elongate flexible body increases from a distal end of the elongate flexible body to a proximal end of the elongate flexible body.

12. A neurovascular catheter comprising:
an elongate flexible body comprising a length of at least about 130 cm, the elongate flexible body comprising:
a distal portion comprising a pre-shaped tip and having a length between about 2 cm and 9 cm,
a transitional portion proximal to the distal portion that is less flexible than the distal portion, wherein the transitional portion has a length of between about 10 cm and about 18 cm; and
a proximal portion proximal to the transitional portion that is less flexible than the transitional portion, wherein the proximal portion has a length between about 110 cm and about 115 cm;
wherein a flexibility profile of the elongate flexible body is measurable with a cantilever beam test with a 5 mm gage length and 4 mm displacement to determine a stiffness measured in a peak load/distance, and wherein:
the stiffness in the distal portion is between about 1 gF and about 200 gF;
the stiffness value in the transitional portion increases from between about 100 gF/mm and about 200 gF/mm to between about 800 gF/mm and about 1000 gF/mm over the length of the transitional portion; and
the stiffness value in the proximal portion is between about 750 gF/mm and about 1150 gF/mm.

13. The neurovascular catheter of claim 12, wherein the elongate flexible body comprises an inner liner, a braid wrapped around inner liner, and a jacket positioned radially outward from the inner line.

14. The neurovascular catheter of claim 13, wherein the inner liner extends and entire length of the elongate flexible body, and wherein the braid ends from about 6 cm to about 8 cm from a distal end of the elongate tubular body.

15. The neurovascular catheter of claim 13, wherein the jacket comprises a plurality of tubular segments having a durometer that decreases in a distal direction.

16. The neurovascular catheter of claim 13, wherein the stiffness in a region spanning the distal portion and the transitional portion increases in a proximal direction from between about 125 gF/mm and about 135 gF/mm to between about 240 gF/mm and about 250 gF/mm over a distance of about 10 mm.

17. The neurovascular catheter of claim 13, wherein the stiffness in a region of the distal portion increases in a proximal direction from between about 75 gF/mm and about 85 gF/mm to between about 125 gF/mm and about 135 gF/mm over a distance of about 40 mm.

18. The neurovascular catheter of claim 13, wherein the stiffness in a region of the distal portion increases in a proximal direction from between about 75 gF/mm and about 85 gF/mm to between about 125 gF/mm and about 135 gF/mm over a distance of about 40 mm.

19. The neurovascular catheter of claim 13, wherein the stiffness of the transitional portion increases in a proximal direction from between about 240 gF/mm and about 250 gF/mm to between about 850 gF/mm and about 860 gF/mm over a distance of about 12 cm.

20. The neurovascular catheter of claim 13, wherein an outer diameter of the elongate flexible body along the distal portion tapers in a distal direction from between about 0.075 in and about 0.085 in to between about 0.055 in and about 0.065 in. over a distance of between about 5.5 cm and about 6.5 cm.
